(19) United States
(12) Patent Application Publication
(10) Pub. No.: US 2024/0390130 A1
(43) Pub. Date: Nov. 28, 2024

(54) VASCULAR GRAFT WITH PULSATION DAMPING

(71) Applicant: ATRIUM MEDICAL CORPORATION, Merrimack, NH (US)

(72) Inventor: Jamie Stephen Henderson, Amherst, NH (US)

(73) Assignee: ATRIUM MEDICAL CORPORATION, Merrimack, NH (US)

(21) Appl. No.: 18/708,453

(22) PCT Filed: Nov. 7, 2022
(86) PCT No.: PCT/US2022/079373

§ 371 Date: May 8, 2024

Related U.S. Application Data
(60) Provisional application No. 63/277,379, filed on Nov. 9, 2021.

(51) Int. Cl. A61F 2/06

(52) U.S. Cl. CPC A61F 2/064; A61F 2230/0006; A61F 2250/0029; A61F 2250/0036

(57) ABSTRACT

A vascular graft includes an implantable tubular body defining a lumen and at least one support frame engaged to the tubular body for modifying a shape of a cross-section of at least a portion of the lumen. The tubular body and at least one support frame are configured to receive pulsatile blood flow so that as pressure of blood flowing through the lumen increases, a cross-sectional area of the portion of the lumen increases while a perimeter of the portion of the lumen remains substantially unchanged in order to dampen the pulsatile blood flow. A method of forming a vascular graft including a step of attaching at least one support frame to a tubular body defining a lumen that modifies a shape of a cross-section of at least a portion of the lumen of the tubular body is also disclosed herein.

### CROSS-REFERENCE TO RELATED APPLICATIONS

This application claims the benefit of priority to U.S. Provisional Patent Application No. 63/277,379, filed Nov. 9, 2021, the contents of which are incorporated herein by reference in its entirety.

### BACKGROUND OF THE DISCLOSURE

### Field of the Disclosure

The present disclosure relates to a vascular graft for connecting body passageways, such as for creating a fluid connection between an artery and a vein, and, more particularly, to a vascular graft including portions configured to absorb, damp, reduce, or counteract forces exerted on the graft due to increases in pressure of fluid flowing through the graft, as occurs from pulsating blood flow.

### Description of Related Art

Procedures for creating fistulas are widely known. A fistula may refer to an opening or channel extending between two lumens of different organs, such as a channel formed by connecting openings extending through a wall of an artery and a wall of a vein for establishing a fluid connection between the artery and vein. For example, a fistula can be created between an artery and vein in an arm of a dialysis patient for providing hemodialysis access for dialysis treatment. In other words, a fistula constitutes an opening or channel extending between lumens of different organs, which enables an abnormal flow of material between the different organs. For example, an arteriovenous fistula formed by the connection of an artery directly to a vein enables an abnormal flow of arterial blood directly into a vein, thereby bypassing any capillary network.

In order to avoid the need to repeatedly puncture artery or vein tissues during dialysis treatment, a graft (referred to herein as a vascular graft or an arterio-venous (AV) shunt graft or as an AV graft) can be implanted to provide a shunt between an artery and a vein. Conventional AV grafts can be implanted into an arm, leg, chest, or another convenient location of a patient. The implanted graft provides a puncture location during dialysis treatments that does not require repeated puncturing of venous or arterial tissue. Typically, the AV graft is of relatively long length to provide maximum length for needle puncturing. Because of the relatively long length, the AV graft typically has a longer length than the spacing between the relevant artery and vein, and, as such, is often bent into a U-shape. For implantation, two relatively shallow channels are subcutaneously “tunneled” into the necessary U-shaped pattern, with a tunneler or guidewire being used to draw the AV graft into proper position. The ends of the graft are sutured, or are otherwise connected to, the selected artery and vein.

Conventional vascular grafts are often formed from bendable tubular structures having a round cross-section formed from a polymeric material, such as polytetrafluoroethylene (PTFE) or expanded polytetrafluoroethylene (ePTFE). In order to maintain structural integrity of the tubular structure of the vascular graft even after being repeatedly punctured during dialysis treatments, the graft is often relatively rigid with respect to its cross-section. Nevertheless, due to the repeated puncturing, AV grafts have a limited life of about 6 to 12 months. After that period of time, structural integrity and/or the sealing ability of the AV graft becomes excessively compromised. Also, the graft may become occluded due to vessel swelling, which begins to occur several months after implantation. Once the structural integrity and/or fluid flow through the graft are compromised, a new AV graft is implanted into the patient at a new location. Patients who require hemodialysis over extensive periods of time may have multiple AV grafts implanted at different body locations.

In addition to being bendable, some conventional vascular grafts are formed from stretchable materials meaning that a length of the graft can increase when ends of the graft are tensioned, as may occur as fluid pressure increases. For example, the GORE-TEX® Stretch Vascular Graft manufactured by W. L. Gore & Associates, Inc. is capable of lengthening by about 10% to 15% from its unbiased or relaxed length. The Stretch Vascular Graft exhibits a significant elastic retraction force when initially tensioned. The Stretch Vascular Graft is not radially compliant meaning that a cross-sectional area of the Stretch Vascular Graft does not substantially increase when fluid pressure increases. The mechanism for creating the semi-elastic behavior of the Stretch Vascular Graft is due to kinking of fibrils in the graft microstructure. This kinking can be created by compressing the graft and heating it to a temperature sufficient to make the fibrils relax and reset into the kinked position. This structure, when tensioned, applies force to the kinked fibrils eventually leading to creep relaxation towards the tensioned position and a loss in elasticity of the material.

### SUMMARY OF THE DISCLOSURE

According to one aspect of the disclosure, a vascular graft includes an implantable tubular body defining a lumen and at least one support frame engaged to the tubular body for modifying a shape of a cross-section of at least a portion of the lumen. The tubular body and at least one support frame are configured to receive pulsatile blood flow so that as pressure of blood flowing through the lumen increases, a cross-sectional area of the portion of the lumen increases while a perimeter of the portion of the lumen remains substantially unchanged in order to dampen the pulsation of the blood flow.

According to another aspect, a method of forming a vascular graft includes a step of attaching at least one support frame to a tubular body defining a lumen that modifies a shape of a cross-section of at least a portion of the lumen of the tubular body. The tubular body and the at least one support frame are configured so that when a pressure of blood flowing through the lumen increases a cross-sectional area of the portion of the lumen increases while a perimeter of the portion of the lumen remains substantially unchanged. The method further includes a step of bending the tubular body or both the tubular body and the at least one support frame to form a vascular graft having parallel substantially linear segments and a u-bend segment connecting the parallel linear segments.

According to another aspect, a vascular graft includes an implantable tubular body having an open first end, an open second end, and a sidewall extending between the first end and the second end, the sidewall defining a lumen. The sidewall of the tubular body includes at least one raised ridge protruding radially outwardly relative to other portions of the sidewall of the tubular body and extending axially from the first end to the second end of the tubular body so as to define a channel of the lumen. A flow path defined by the channel spirals about a central longitudinal axis of the tubular body.

According to another aspect, a vascular graft includes an implantable tubular body having an open first end, an open second end, and a sidewall extending between the first end and the second end, the sidewall defining a lumen. The tubular body is configured such that as pressure of blood flowing through the lumen increases a cross-sectional area of a portion of the lumen increases while a perimeter of the portion of the lumen remains substantially unchanged.

According to another aspect, a vascular graft includes an implantable tubular body having a wall defining a lumen, wherein the wall has a variable non-concentric wall thickness and includes at least one thin portion and at least one thick portion, wherein the tubular body is configured to receive pulsatile blood flow so that as pressure of blood flowing through the lumen increases, a cross-sectional area of the lumen increases while a perimeter of the lumen remains substantially unchanged in order to dampen pulsation of the blood flow.

According to another aspect, a vascular graft includes an implantable tubular body having a wall defining a lumen. The wall has a variable wall thickness and includes at least one thin portion and at least one thick portion. The tubular body is configured to receive pulsatile blood flow so that as pressure of blood flowing through the lumen increases, the at least one thin portion of the wall moves radially outward in order to dampen pulsation of the pulsatile blood flow.

Non-limiting illustrative examples of embodiments of the present disclosure will now be described in the following numbered clauses:

Clause 1: A vascular graft comprising: an implantable tubular body defining a lumen; and at least one support frame engaged to the tubular body for modifying a shape of a cross-section of at least a portion of the lumen, wherein the tubular body and at least one support frame are configured to receive pulsatile blood flow so that as pressure of blood flowing through the lumen increases, a cross-sectional area of the portion of the lumen increases while a perimeter of the portion of the lumen remains substantially unchanged in order to dampen pulsation of the blood flow.

Clause 2: The vascular graft of clause 1, wherein the tubular body and the at least one support frame are configured to transition between a first state, in which the portion of the lumen has a first cross-sectional area and a first perimeter and a second state, in which the portion of the lumen has a second cross-sectional area and a second perimeter, and wherein the second cross-sectional area is greater than the first cross-sectional area and the second perimeter is substantially equivalent to the first perimeter, and radial compliance of the tubular body permits transition between the first state and the second state.

Clause 3: The vascular graft of clause 2, wherein the tubular body and the at least one support frame are configured to be in the first state when pressure of fluid flowing through the lumen is diastolic pressure for a patient in which the vascular graft is implanted, and the tubular body and the at least one support frame are configured to transition to the second state when pressure of the fluid flowing through the lumen increases to systolic pressure for the patient in which the vascular graft is implanted.

Clause 4: The vascular graft of clause 2 or clause 3, wherein the tubular body and at least one support frame are configured to be in the first state when the pressure is less than a threshold pressure, and in the second state when the pressure is greater than or equal to the threshold pressure.

Clause 5: The vascular graft of any of clauses 2 to 4, wherein in the first state, the shape of the cross-section of the lumen is elongated having a major dimension that is substantially greater than a minor dimension of the shape, and wherein in the second state, the shape of the cross-section of the lumen is substantially round with the minor dimension approaching or being substantially equivalent in length to the major dimension.

Clause 6: The vascular graft of any of clauses 2 to 5, wherein in the first state, the shape of the cross-section of the lumen is an oval, and in the second state, the shape of the cross-section of the lumen is a circle.

Clause 7: The vascular graft of any of clauses 2 to 6, wherein the second cross-sectional area is at least about 10% greater than the first cross-sectional area.

Clause 8: The vascular graft of any of clauses 2 to 7, wherein the second perimeter is no more than about 5%, preferably about 2% to about 3%, greater than the first perimeter.

Clause 9: The vascular graft of any of clauses 2 to 8, wherein the second cross-sectional area is from about 10% greater to about 15% greater than the first cross-sectional area.

Clause 10: The vascular graft of any of clauses 1 to 9, wherein the tubular body and the at least one support frame are configured to dampen pulsation of the blood flow through the lumen of the tubular body compared to when no support frame is present.

Clause 11: The vascular graft of clause 10, wherein a pressure change for the pulsatile blood flow through the vascular graft is reduced by 5% to 20%, or by 5% to 50%, or by 20% to 50%, or by 20% to 90%, or by 50% to 90% compared to when no support frame is present.

Clause 12: The vascular graft of any of clauses 1 to 11, wherein an outer surface of the tubular body comprises a low-friction material.

Clause 13: The vascular graft of any of clauses 1 to 12, wherein the tubular body comprises at least one of ePTFE or silicone.

Clause 14: The vascular graft of any of clauses 1 to 13, wherein the graft is an arterio-venous shunt graft.

Clause 15: The vascular graft of any of clauses 1 to 14, wherein the at least one support frame comprises at least one of a band, a spring, a clip, or a helical coil configured to compress one or more portions of the tubular body when pressure of blood in the lumen is low, and wherein as the pressure of blood in the lumen increases these one or more portions of the tubular body expand radially outwardly against compression exerted by the at least one support frame.

Clause 16: The vascular graft of any of clauses 1 to 14, wherein the at least one support frame comprises at least one of a band, a spring, a clip, or a helical coil configured to radially expand one or more portions of the tubular body when pressure of blood in the lumen is low causing the shape of the cross-section of the lumen to be oblong, and wherein as the pressure of blood in the lumen increases other portions of the tubular body expand radially outwardly causing the shape of the cross-section of the lumen to become more round.

Clause 17: The vascular graft of any of clauses 1 to 14, wherein the at least one support frame comprises axially extending members forming an interconnected first helix and second helix.

Clause 18: The vascular graft of any of clauses 1 to 14, wherein the at least one support frame comprises a heat-set metallic coil defining a lumen with an oval cross-section at body temperature, in an unbiased state, and configured to engage portions of an outer surface of the tubular body to compress the tubular body at body temperature.

Clause 19: The vascular graft of clause 18, wherein as pressure of blood flow through the lumen of the tubular body increases, the shape of the cross-section of the tubular body becomes more round, causing the cross-section of the lumen of the metallic coil to become more round.

Clause 20: The vascular graft of any of clauses 1 to 14, wherein the at least one support frame comprises a heat-set metallic coil defining a lumen with a substantially circular cross-section at body temperature, in an unbiased state, and configured to engage portions of an outer surface of the tubular body to expand the tubular body at body temperature.

Clause 21: The vascular graft of clause 20, wherein as pressure of blood flow through the lumen of the tubular body increases, the shape of the cross-section of the tubular body becomes more round, causing a shape of the cross-section of the lumen of the metallic coil to become an almost round oval.

Clause 22: The vascular graft of any of clauses 1 to 14, wherein the at least one support frame is fused to portions of an outer surface of the tubular body.

Clause 23: The vascular graft of any of clauses 1 to 14, wherein the at least one support frame is fixedly connected to an outer surface of the tubular body by an adhesive.

Clause 24: The vascular graft of any of clauses 1 to 14, wherein the at least one support frame connects to the tubular body along a major dimension of the tubular body, and wherein the outer surface of the tubular body is spaced apart from the at least one support frame at other portions of the outer surface.

Clause 25: The vascular graft of any of clauses 1 to 14, wherein the at least one support frame comprises an elastomeric sleeve, and wherein the tubular body is inserted in and directly or indirectly connected to an inner surface of the elastomeric sleeve.

Clause 26: The vascular graft of clause 25, wherein the elastomeric sleeve defines a lumen, and wherein a shape of a cross-section of at least a portion of the lumen of the elastomeric sleeve is an oval at low pressure and becomes increasingly round as pressure increases in the lumen of the tubular body, which causes the cross-section of the lumen of the elastomeric sleeve to become round.

Clause 27: The vascular graft of clause 25 or clause 26, wherein the elastomeric sleeve is connected to the tubular body by at least one of adhesive bonding, solvent bonding, hot melt bonding, or sintering.

Clause 28: The vascular graft of any of clauses 1 to 14, wherein the tubular body comprises an extruded tube and the at least one support frame comprises one or more polymeric layers fused to an outer surface of the tubular body.

Clause 29: The vascular graft of any of clauses 1 to 14, wherein the tubular body comprises at least one raised ridge extending axially along at least a portion of the tubular body defining a channel of the lumen that spirals about a central longitudinal axis of the tubular body.

Clause 30: The vascular graft of clause 29, wherein the tubular body comprises multiple raised ridges extending axially along the portion of the tubular body defining multiple channels of the lumen.

Clause 31: The vascular graft of clause 4, wherein the threshold pressure is about 90 mmHg.

Clause 32: The vascular graft of any of clauses 1 to 31, wherein the tubular body comprises an annular sidewall extending between a first end and a second end of the tubular body, and wherein, for at least a portion of the tubular body, a thickness of the sidewall varies about a periphery of the tubular body.

Clause 33: The vascular graft of clause 32, wherein thinner portions of the sidewall of the tubular body provide radial compliance for the tubular body.

Clause 34: The vascular graft of any of clauses 1 to 33, wherein a sidewall of the tubular body has a non-concentric wall thickness where a central axis of the lumen is not co-extensive with a central axis of the tubular body.

Clause 35: A method of forming a vascular graft comprising the steps of: attaching at least one support frame to a tubular body defining a lumen that modifies a shape of a cross-section of at least a portion of the lumen of the tubular body, wherein the tubular body and the at least one support frame are configured so that when a pressure of blood flowing through the lumen increases a cross-sectional area of the portion of the lumen increases while a perimeter of the portion of the lumen remains substantially unchanged; and bending the tubular body or both the tubular body and the at least one support frame to form a vascular graft having substantially parallel substantially linear segments and a u-bend segment connecting the substantially parallel substantially linear segments.

Clause 36: The method of clause 35, wherein when the at least one support frame is attached to the tubular body, the tubular body and at least one support frame are configured to conform to a first state when pressure of blood flowing through the lumen is diastolic pressure for a patient in which the vascular graft is implanted, and the tubular body and at least one support frame are configured to transition to a second state when pressure of the blood flowing through the lumen increases to systolic pressure for the patient in which the vascular graft is implanted.

Clause 37: The method of clause 35 or clause 36, wherein the tubular body and the at least one support frame are configured to be in the first state when the pressure is less than a threshold pressure and in the second state when the pressure is greater than or equal to the threshold pressure.

Clause 38: The method of clause 37, wherein the threshold pressure is about 90 mmHg.

Clause 39: The method of any of clauses 36 to 38, wherein a cross-sectional area of the lumen in the second state is about 10% greater to about 15% greater than the cross-sectional area of the lumen in the first state.

Clause 40: The method of any of clauses 35 to 39, wherein the at least one support frame comprises at least one of a band, a spring, a clip, or a helical coil that, when attached to the tubular body, compresses one or more portions of the tubular body when pressure of blood in the lumen is low, and wherein as the pressure of blood in the lumen increases portions of the tubular body expand radially outwardly against a compression force exerted by the at least one support frame.

Clause 41: The method of any of clauses 35 to 39, wherein the at least one support frame comprises at least one of a band, a spring, a clip, or a helical coil that, when attached to the tubular body, causes one or more portions of the tubular body to radially expand when pressure of blood in the lumen is low causing the shape of the cross-section of the lumen to be oblong, and wherein as the pressure of blood in the lumen increases, other portions of the tubular body expand radially outwardly causing the shape of the cross-section of the lumen to become more round.

Clause 42: The method of any of clauses 35 to 39, wherein attaching the at least one support frame to the tubular body comprises fusing the at least one support frame to one or more portions of an outer surface of the tubular body.

Clause 43: The method of any of clauses 35 to 39, wherein attaching the at least one support frame to the tubular body comprises fixedly attaching the at least one support frame to one or more portions of an outer surface of the tubular body by an adhesive.

Clause 44: The method of any of clauses 35 to 39, wherein attaching the at least one support frame to the tubular body comprises attaching the at least one support frame to the tubular body at points along a major diameter of the tubular body, so that other portions of the at least one support frame are spaced apart from the outer surface of the tubular body.

Clause 45: The method of any of clauses 35 to 39, further comprising attaching a first end of the vascular graft to a vein and a second end of the vascular graft to an artery to permit blood flow from artery to vein through the lumen of the tubular body, or attaching the first end of the vascular graft to an artery and the second end of the vascular graft to another artery to permit blood flow from artery to artery through the lumen of the tubular body.

Clause 46: The method of clause 45, wherein the vascular graft connected between the vein and the artery is configured for dialysis treatment.

Clause 47: A vascular graft comprising: an implantable tubular body comprising an open first end, an open second end, and a sidewall extending between the first end and the second end, the sidewall defining a lumen, wherein the sidewall of the tubular body comprises at least one raised ridge protruding radially outwardly relative to other portions of the sidewall of the tubular body and extending axially from the first end to the second end of the tubular body so as to define a channel of the lumen, and wherein a flow path defined by the channel spirals about a central longitudinal axis of the tubular body.

Clause 48: The vascular graft of clause 47, further comprising at least one support frame connected to at least a portion of the sidewall of the tubular body that compresses at least a portion of the sidewall to form the at least one raised ridge.

Clause 49: The vascular graft of clause 47 or clause 48, wherein the tubular body comprises multiple raised ridges extending axially along the tubular body so as to define multiple channels of the lumen.

Clause 50: The vascular graft of clause 49, wherein the multiple ridges and channels are positioned to impart a spiral flow for blood flowing through the tubular body.

Clause 51: The vascular graft of clause 49 or clause 50, wherein flow paths defined by each of the multiple channels complete at least one full rotation about the perimeter of the tubular body between the first end and the second end of the tubular body.

Clause 52: The vascular graft of any of clauses 47 to 51, wherein in a first state, a shape of the cross-section of the lumen is a star having points and recessed portions, and as pressure of blood flowing through the lumen increases, the vascular graft transitions to a second state in which the recessed portions of the star move radially outwardly away from the central longitudinal axis of the tubular body, thereby causing the shape of the cross-section of the lumen to become more round.

Clause 53: The vascular graft of clause 52, wherein portions of the tubular body forming recessed portions of the star-shaped lumen are more flexible than portions of the tubular body forming points of the star-shaped lumen.

Clause 54: The vascular graft of clause 52 or clause 53, wherein portions of the tubular body forming recessed portions of the star-shaped lumen are thinner than portions of the tubular body forming points of the star-shaped lumen.

Clause 55: The vascular graft of any of clauses 52 to 54, wherein the tubular body is in the first state when pressure of blood flowing through the lumen is less than a threshold blood pressure and in the second state when the pressure of blood is greater than the threshold blood pressure.

Clause 56: The vascular graft of clause 55, wherein the threshold blood pressure is about 90 mmHg.

Clause 57: The vascular graft of any of clauses 52 to 56, wherein a cross-sectional area of the lumen when the graft is in the second state is from about 10% greater to about 15% greater than the cross-sectional area of the lumen when the graft is in the first state.

Clause 58: The vascular graft of any of clauses 47 to 57, wherein, for at least a portion of the tubular body, a thickness of the sidewall varies about a periphery of the tubular body.

Clause 59: The vascular graft of clause 58, wherein thinner portions of the sidewall of the tubular body provide radial compliance for the tubular body.

Clause 60: The vascular graft of any of clauses 47 to 59, wherein the sidewall of the tubular body has a non-concentric wall thickness where a central axis of the lumen is not co-extensive with a central axis of the tubular body.

Clause 61: A vascular graft comprising: an implantable tubular body comprising an open first end, an open second end, and a sidewall extending between the first end and the second end, the sidewall defining a lumen, wherein the tubular body is configured such that as pressure of blood flowing through the lumen increases a cross-sectional area of a portion of the lumen increases while a perimeter of the portion of the lumen remains substantially unchanged.

Clause 62: The vascular graft of clause 61, wherein the tubular body is configured to transition between a first state in which the portion of the lumen has a first cross-sectional area and a first perimeter, and a second state in which the portion of the lumen has a second cross-sectional area and a second perimeter, and wherein the second cross-sectional area is greater than the first cross-sectional area and the second perimeter is substantially equivalent to the first perimeter, and radial compliance of the tubular body permits transition between the first state and the second state.

Clause 63: The vascular graft of clause 62, wherein the tubular body is configured to be in the first state when pressure of blood flowing through the lumen is diastolic pressure for a patient in which the vascular graft is implanted, and the tubular body is configured to transition to the second state when pressure of the blood flowing through the lumen increases to systolic pressure for the patient in which the vascular graft is implanted.

Clause 64: The vascular graft of any of clauses 61 to 63, wherein the tubular body is in the first state when the pressure of the blood is less than a threshold pressure and in the second state when the pressure of the blood is greater than or equal to the threshold pressure.

Clause 65: The vascular graft of clause 64, wherein the threshold blood pressure is about 90 mmHg.

Clause 66: The vascular graft of any of clauses 62 to 65, wherein in the first state, the shape of the cross-section of the lumen is selected from the group consisting of a triangle, star, diamond, rectangle, square, polygon, trapezoid, crescent, oval, or elongated pill shape.

Clause 67: The vascular graft of any of clauses 62 to 65, wherein in the first state, the shape of the cross-section of the lumen is an irregular shape.

Clause 68: The vascular graft of any of clauses 61 to 67, wherein the tubular body comprises at least one raised ridge extending axially along at least a portion of the tubular body so as to define a channel of the lumen that spirals about a central longitudinal axis of the tubular body.

Clause 69: The vascular graft of clause 68, wherein the tubular body comprises multiple raised ridges extending axially along the tubular body so as to define multiple channels of the lumen.

Clause 70: The vascular graft of any of clauses 61 to 69, wherein, for at least a portion of the tubular body, a thickness of the sidewall varies about a periphery of the tubular body.

Clause 71: The vascular graft of clause 70, wherein thinner portions of the sidewall of the tubular body provide radial compliance for the tubular body.

Clause 72: The vascular graft of any of clauses 61 to 71, wherein the sidewall of the tubular body has a non-concentric wall thickness where a central axis of the lumen is not co-extensive with a central axis of the tubular body.

Clause 73: A vascular graft comprising: an implantable tubular body having a wall defining a lumen, wherein the wall has a variable non-concentric wall thickness and includes at least one thin portion and at least one thick portion, wherein the tubular body is configured to receive pulsatile blood flow so that as pressure of blood flowing through the lumen increases, a cross-sectional area of the lumen increases while a perimeter of the lumen remains substantially unchanged in order to dampen pulsation of the blood flow.

Clause 74: The vascular graft of clause 73, wherein the at least one thin portion possesses about 10-90% of the thickness of the at least one thick portion of the wall, or the thin portion possesses about 20-80% of the thickness of the at least one thick portion of the wall, or the at least one thin portion possesses about 10-50% of the thickness of the at least one thick portion of the wall.

Clause 75: The vascular graft of clause 73, wherein when in a relaxed low pressure state a cross-sectional configuration of the tubular body has a crescent moon shape and when in a flexed high pressure state the cross-sectional configuration of the tubular body has a round or almost round shape.

Clause 76: A vascular graft comprising: an implantable tubular body having a wall defining a lumen, wherein the wall has a variable wall thickness and includes at least one thin portion and at least one thick portion, wherein the tubular body is configured to receive pulsatile blood flow so that as pressure of blood flowing through the lumen increases, the at least one thin portion of the wall moves radially outward in order to dampen pulsation of the pulsatile blood flow.

Clause 77: The vascular graft of clause 76, wherein the implantable tubular body is configured to adopt an out-of-round cross-sectional shape as the at least one thin portion of the wall moves radially outward, thereby dampening the pulsation of the blood flow.

Clause 78: The vascular graft of clause 76 or clause 77, wherein the implantable tubular body comprises a first thin portion on a first side of the wall and a second thin portion on an opposing side of the wall.

Clause 79: The vascular graft of any of clauses 76-78, wherein the implantable tubular body is configured to transition from a relaxed low pressure state to a high pressure state, and wherein a cross-sectional shape of a lumen defined by the tubular body becomes less round as the tubular body transitions from the relaxed low pressure state to the high pressure state.

Clause 80: The vascular graft of clause 79, wherein the cross-sectional shape of the lumen in the relaxed low pressure state is substantially circular and the cross-sectional shape in the high pressure state is substantially an oval shape.

Clause 81: The vascular graft of any of clauses 76-80, wherein the at least one thin portion possesses about 10-90% of the thickness of the at least one thick portion of the wall, or the thin portion possesses about 20-80% of the thickness of the at least one thick portion of the wall, or the at least one thin portion possesses about 10-50% of the thickness of the at least one thick portion of the wall.

These and other features and characteristics of the invention, as well as the methods of operation and functions of the related elements of structures and the combination of parts and economies of manufacture, will become more apparent upon consideration of the following description and appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and the claims, the singular form of “a”, “an”, and “the” include plural referents unless the context clearly dictates otherwise.

### BRIEF DESCRIPTION OF THE DRAWINGS

FIG. 1 is a schematic drawing of a top view of a vascular graft according to an example of the present disclosure;

FIG. 2A is a schematic drawing of a cross-sectional view of the graft of FIG. 1 taken along line 2A-2A in a low pressure state;

FIG. 2B is a schematic drawing of a cross-sectional view of the graft of FIG. 1 taken along line 2A-2A in a high pressure state;

FIG. 3A is a schematic drawing of a cross-sectional view of another example of a vascular graft in a low pressure state;

FIG. 3B is a schematic drawing of another cross-sectional view of the vascular graft of FIG. 3A in a high pressure state;

FIGS. 3C-3F are schematic drawings of side views of the vascular graft of FIG. 3A showing a surge or wave of fluid passing through the graft;

FIG. 4A is a schematic drawing of a cross-sectional view of another example of a vascular graft in a low pressure state;

FIG. 4B is a schematic drawing of another cross-sectional view of the vascular graft of FIG. 4A showing the graft in a high pressure state;

FIG. 4C is a schematic drawing of a top view of the vascular graft of FIG. 4A showing the low pressure state;

FIG. 5 is a flow chart showing a method of forming a vascular graft according to an example of the present disclosure;

FIG. 6A is a schematic drawing of a cross-sectional view of another example of a vascular graft in a low pressure state;

FIG. 6B is a schematic drawing of another cross-sectional view of the vascular graft of FIG. 6A showing the graft in a high pressure state;

FIG. 7A is a schematic drawing of a cross-sectional view of another example of a vascular graft in a low pressure state;

FIG. 7B is a schematic drawing of another cross-sectional view of the vascular graft of FIG. 7A showing the graft in a high pressure state;

FIG. 7C is a schematic drawing of a top view of the vascular graft of FIG. 7A showing the low pressure state;

FIG. 8A is a schematic drawing of a cross-sectional view of another example of a vascular graft in a low pressure state comprising a single-layer tubular body;

FIG. 8B is a schematic drawing of a cross-sectional view of another example of a vascular graft in a low pressure state comprising a multi-layer tubular body;

FIG. 9A is a schematic drawing of cross-sectional views of other configurations of vascular grafts in a low pressure state;

FIG. 9B is a schematic drawing of corresponding cross-sectional views of the other configurations of the vascular graft of FIG. 9A showing the graft configuration in a high pressure state;

FIG. 9C is a schematic drawing of a cross-sectional view of another configuration of a vascular graft in a low pressure state;

FIG. 9D is a schematic drawing of a corresponding cross-sectional view of the vascular graft of FIG. 9C in a high pressure state;

FIG. 9E is a schematic drawing of a cross-sectional view of another configuration of a vascular graft in a low pressure state;

FIG. 9F is a schematic drawing of a corresponding cross-sectional view of the vascular graft of FIG. 9E in a high pressure state;

FIG. 9G is a schematic drawing of a cross-sectional view of another configuration of a vascular graft in a low pressure state;

FIG. 9H is a schematic drawing of a corresponding cross-sectional view of the vascular graft of FIG. 9G in a high pressure state;

FIG. 10A is a schematic drawing of a side view of another example of a vascular graft in a low pressure state;

FIG. 10B is a schematic drawing of a cross-sectional view of the vascular graft of FIG. 10A taken along line 10-10 in the low pressure state; and

FIG. 10C is a schematic drawing of another cross-sectional view of the vascular graft of FIG. 10A taken along line 10-10 showing the graft in a high pressure state.

### DETAILED DESCRIPTION

The illustrations generally show illustrative and non-limiting aspects of the devices, assemblies, and methods of the present disclosure. While the descriptions present various aspects of the devices and assemblies, it should not be interpreted in any way as limiting the disclosure. Furthermore, modifications, concepts, and applications of the disclosure's aspects are to be interpreted by those skilled in the art as being encompassed by, but not limited to, the illustrations and descriptions herein.

Further, for purposes of the description hereinafter, the terms “end”, “upper”, “lower”, “right”, “left”, “vertical”, “horizontal”, “top”, “bottom”, “lateral”, “longitudinal”, “radial”, and derivatives thereof shall relate to the disclosure as it is oriented in the drawing figures. The term “proximal” refers to the direction toward the center or central region of the device. The term “distal” refers to the outward direction extending away from the central region of the device. However, it is to be understood that the disclosure may assume various alternative variations and step sequences, except where expressly specified to the contrary. It is also to be understood that the specific devices and processes illustrated in the attached drawings, and described in the following specification, are simply exemplary aspects of the disclosure. Hence, specific dimensions and other physical characteristics related to the aspects disclosed herein are not to be considered as limiting. For the purpose of facilitating understanding of the disclosure, the accompanying drawings and description illustrate preferred aspects thereof, from which the disclosure, various aspects of its structures, construction and method of operation, and many advantages may be understood and appreciated.

With reference to the figures, a vascular graft 10, 210 comprises or is formed from an implantable tubular body 12, 212 defining a lumen 14, 214 configured to permit a flow of a body fluid, such as a pulsating blood flow, through the lumen 14, 214. The vascular graft 10, 210 can be configured to be connected between body passageways, such as between an artery and a vein, to establish fluid flow between the artery and the vein. When used for dialysis treatments, the vascular graft 10, 210 (also referred to as an arterio-venous shunt graft) is implanted, for example, in an arm, leg, chest wall, or another convenient location of a patient's body. As used herein, a “patient” refers to a human patient, though it is understood that the vascular grafts disclosed herein may be adapted for use with other mammals and/or other organisms. The implanted graft 10, 210 is configured to be punctured by a needle cannula during the dialysis treatment.

Implanted vascular grafts are known to fail (often within from about 6 and 12 months) due to intimal hyperplasia (e.g., vessel swelling and/or abnormal cellular accumulation resulting in eventual occlusion of the lumen of the vascular graft). While there are many theories about root causes of intimal hyperplasia, it is believed that at least one contributing factor can be pulsatile, high pressure blood entering the more delicate vein, causing damage to vein tissues, thereby resulting in intimal hyperplasia as a response to this damage. In some cases, flow rate for pulsatile blood entering the vein can be controlled by banding the graft upstream and booting the downstream anastomosis. However, banding the graft does not address pulsation, but merely reduces the overall flow rate and pressure.

Conventional vascular grafts, such as grafts comprising polymeric materials (e.g., polytetrafluoroethylene (PTFE) or expanded polytetrafluoroethylene (ePTFE)), are formed to be substantially inelastic (e.g., to avoid stretching resulting in axial and/or radial expansion) and have a round cross-section. Thus, a cross-sectional area of the lumen of a conventional graft does not intermittently increase with pulsatile blood flow in any substantial way because the graft is formed from relatively inelastic materials and does not stretch radially outwardly as internal luminal pressure varies with the pulsatile blood flow. In some cases, there may be a very small amount of axial stretch flexibility for some conventional grafts, such as with the previously described GORE-TEX® Stretch Vascular Graft. This small degree of axial stretch flexibility possessed by these grafts does not substantially compensate for pulsation by lengthening the graft because the amount of corresponding increased volume enclosed by the graft due to the axial stretch is inconsequential. Consequently, there is no substantial compensation effect with respect to pulsatile flow because intimal hyperplasia occurs with these grafts as well as those possessing no axial stretch and there is no evidence that grafts possessing trivial degrees of axial stretch can prevent or otherwise effect intimal hyperplasia in any way in attached blood vessels when employed as an arterio-venous (AV) shunt graft.

In the final analysis, the relatively rigid construction of conventional AV grafts in the radial direction is believed to cause fluid (e.g., blood) to accelerate through such a relatively radially rigid graft during a pressure increase wave as occurs with pulsatile flow, thereby causing the fluid to jet into the vein at a higher velocity and/or pressure than normal for the vein. It is believed that this accelerated velocity fluid flow can amplify damage to the vein, potentially leading to intimal hyperplasia as a result of pulsatile blood flow. In contrast, natural blood vessels stretch radially outwardly to compensate for changes in fluid pressure within these blood vessels.

In order to counteract pressure changes in pulsating blood flow, the vascular grafts 10, 210 disclosed herein are configured to undergo changes in shape and/or changes in cross-sectional area of portions of the vascular graft 10 in order to dampen pulsation of the pulsating blood flow. For example, vascular grafts 10 of the present disclosure can have a variable cross-sectional geometry capable of transitioning from an oval or another elongated or compressed shape (i.e., a non-expanded configuration) to a rounder shape having a larger cross-sectional area (i.e., an expanded configuration). It is believed that this extra volume expansion capacity acts as a damper to compensate for pressure waves and reduces the pulsation effect downstream as the change in graft configuration between the non-expanded configuration and the expanded configuration may absorb kinetic energy from pulsatile blood flow. For example, the graft 10, 210 can be configured to reduce a pressure change of pulsatile flow of a fluid through the graft 10, 210 by about 5% to 20%, or by 5% to 50%, or by 20% to 50%, or by 20% to 90%, or, preferably, by about 50% to 90% compared to when no support frame or another structure for supporting the tubular body of the graft 10, 210 is present. In some examples, the vascular graft 10, 210 can be radially compliant having a variable cross-sectional geometry along its entire length, meaning that the graft 10, 210 acts as a damper along its entire length. In other examples, some sections of the graft 10, 210 can have a variable cross-sectional geometry and act as a damper for pressure waves, while other sections of the graft 10, 210 are more rigid and resistant to radial expansion.

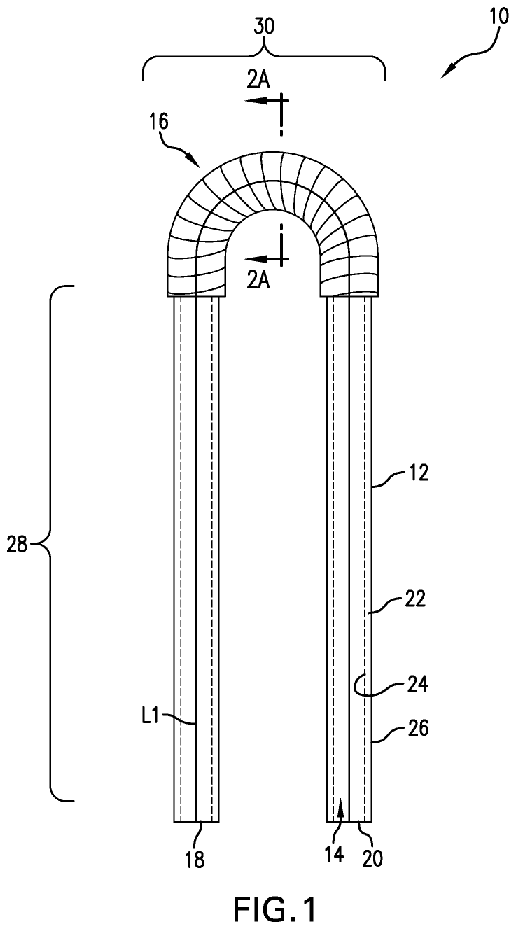

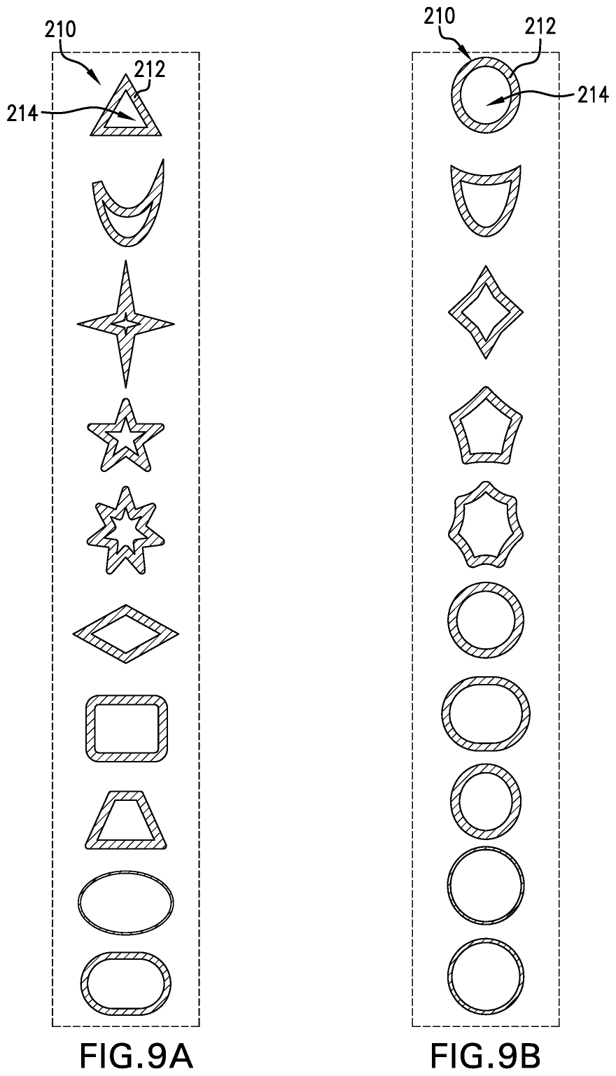

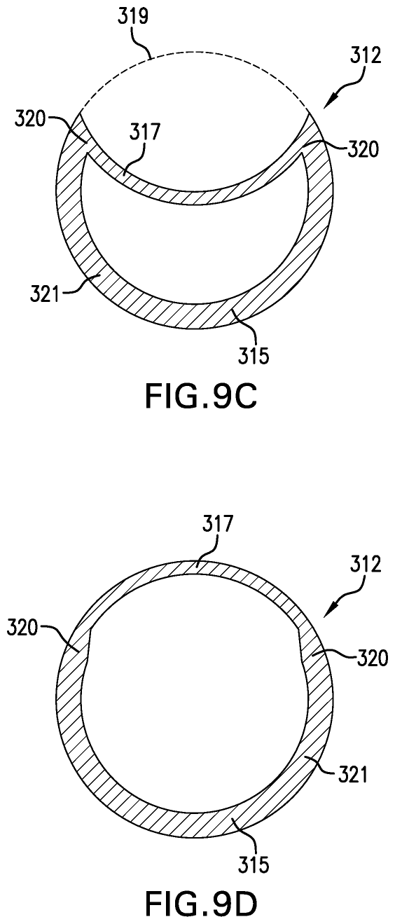

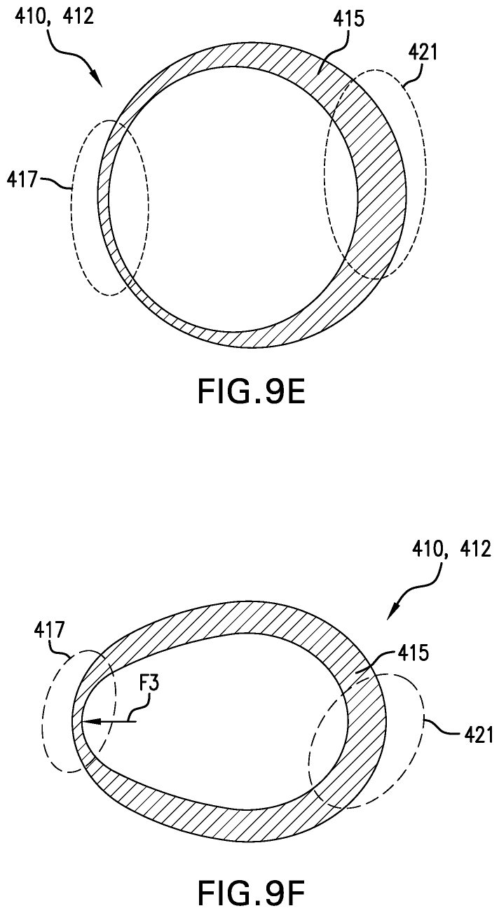

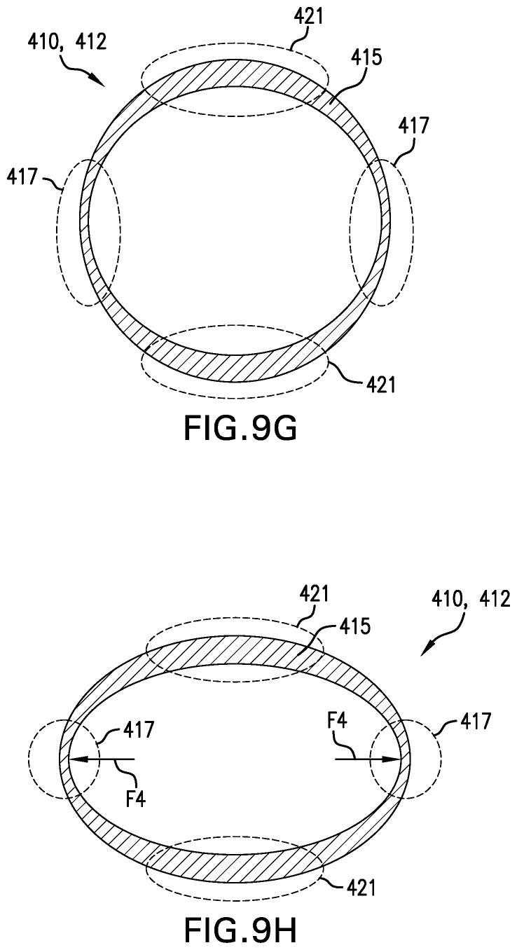

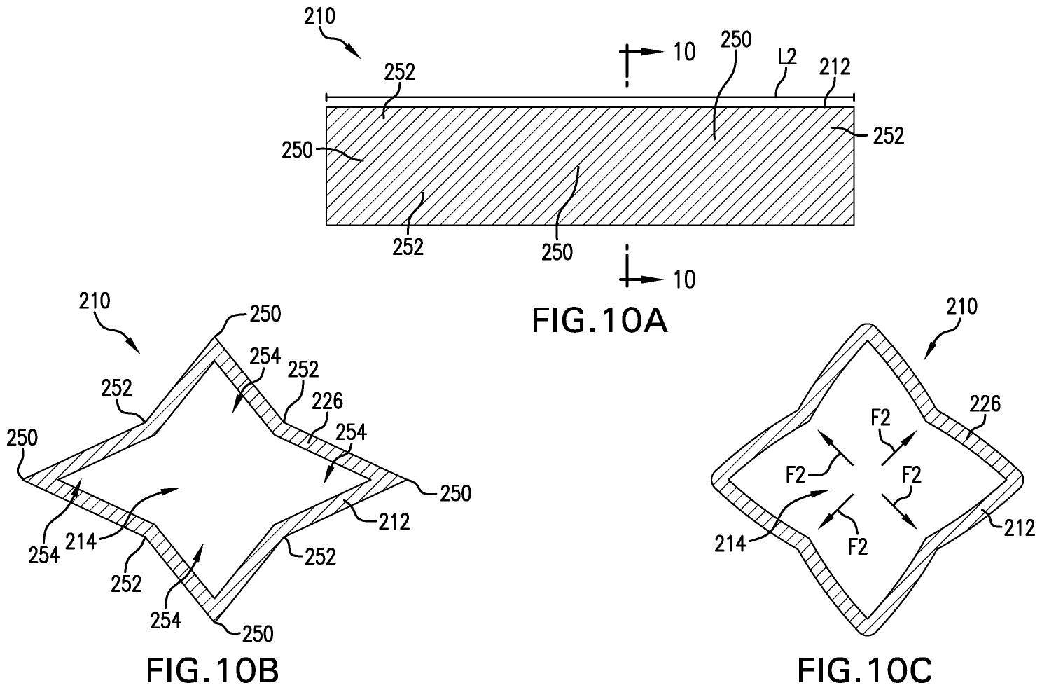

Further, in some examples, as described in connection with FIGS. 1-8, the vascular graft 10 includes support structures for modifying the cross-sectional area of the tubular body 12 when pressure of fluid flowing through the graft 10 is low and that allows the tubular body 12 to expand (e.g., to become more round) as fluid pressure increases. In other examples, as described in connection with FIGS. 9A-10C, the tubular body 212 of the vascular graft 210 is shaped or inherently formed to transition between the compressed or non-expanded low pressure state and the round, rounder, or expanded higher pressure state without any external support or frame.

An exemplary vascular graft 10 including support structures, referred to as a support frame 16, for providing variable cross-sectional geometry is shown in FIG. 1. The vascular graft 10 comprises the implantable tubular body 12 defining the lumen 14 and the support frame 16 engaged to the tubular body 12 for modifying a shape of a cross-section of at least a portion of the lumen 14 either by compressing the tubular body 12, permitting expansion of the tubular body 12, or causing expansion of such portion of the tubular body 12. As shown in FIG. 1, the support frame 16 encloses only a portion of the tubular body 12; however, this configuration is not intended to be limiting. In other examples, the support frame 16 may enclose the tubular body 12 along the entire length of the tubular body 12. In still other examples, the graft 10 can comprise multiple, spaced-apart support frames 16 enclosing different portions of the tubular body 12.

The tubular body 12 can be formed of materials commonly used for forming vascular prostheses, such as textile materials (e.g., polyethylene terephthalate (PET)), polymeric materials (e.g., expanded polytetrafluoroethylene (ePTFE) or silicone), or composites thereof. In some examples, an outer surface 26 of the tubular body 12 may comprise and/or be covered by a low-friction material to reduce or prevent, for example, adhesion of body tissues to the tubular body 12, as such adhesions may adversely affect radial movement of the tubular body 12.

Generally, the material of the tubular body 12 is substantially rigid. As used herein with respect to the tubular body 12, “substantially rigid” means that the tubular body 12 is formed from materials that do not stretch or contract radially or that only stretch or contract radially by a small amount in response to physiologic pressure changes of fluid, such as blood, flowing through the tubular body 12. For example, the tubular body 12 may define a circumference or perimeter (e.g., a distance around the cross-section of the tubular body 12) of a fixed dimension or a maximum obtainable dimension that does not substantially increase or decrease in response to the physiologic pressure changes of the fluid flowing through the tubular body 12. Instead, the geometry, shape, and/or configuration of the tubular body 12 and/or support frame 16 (rather than the material of the tubular body 12) are selected to allow the cross-sectional area of the lumen 14 to increase as physiologic pressure of fluid flowing through the lumen 14 increases, while the dimension (i.e. length) of the perimeter remains fixed. For example, the perimeter (e.g., distance around the cross-section) of the tubular body 12 may change by less than about 5% or, preferably, by from about 2% to about 3% as the graft 10 is exposed to pulsatile blood flow at normal pressures.

In other words, the material of the tubular body 12 is flexible enough so the tubular body 12 may assume a different geometry, shape, and/or configuration in response to physiological pressure changes within the lumen 14 of the tubular body 12, while the perimeter of the tubular body 12 remains constant, meaning that the perimeter neither stretches nor contracts. Conceptually, the perimeter of the tubular body 12 behaves like a loop of string that cannot stretch or contract. The loop of string defines the perimeter of constant length although the loop may assume different geometries, shapes, and/or configurations having substantially different cross-sectional areas, such as an oval or a circle. It is noted, however, that the tubular body may be bendable, so that the tubular body 12 may be bent to the u-shaped configuration shown in FIG. 1.

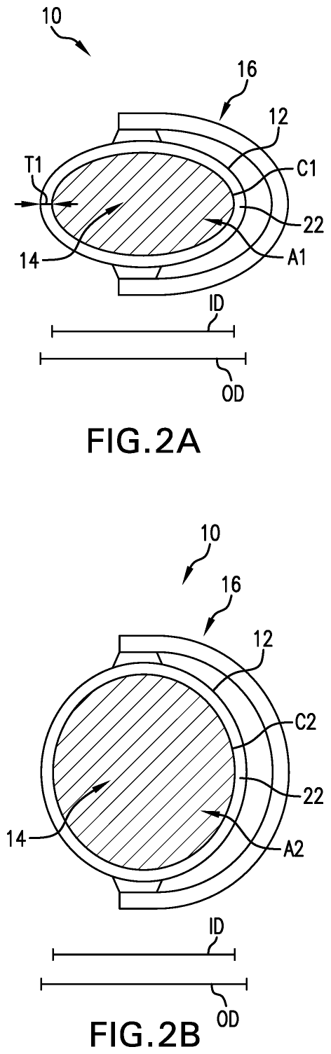

In some examples, the tubular body 12 comprises an open first or arterial end 18, which can be configured to be connected to an artery, an opposing open second or venous end 20, which can be configured to be connected to a vein, and a sidewall 22 extending between the first end 18 and the second end 20 having an inner surface 24 and an outer surface 26, wherein the sidewall 22 defines the lumen 14 that extends the length L1 of the tubular body 12. The graft 10 can be provided in any suitable shape or configuration based on the intended deployment location in the body. In some examples, such as shown in FIG. 1, the graft 10 is u-shaped comprising substantially straight parallel segments 28 with a curved u-shaped segment 30 disposed between the straight segments 28. The tubular body 12 can be any convenient size based on the intended deployment location and fluid volume and/or flow rate for blood that flows through the lumen 14. For example, the tubular body 12 of the graft 10 can have a length L1 of from about 10 cm to about 100 cm. The sidewall 22 of the tubular body 12 can have a thickness T1 (shown in FIG. 2A) of from about 0.5 mm to about 1.5 mm. The inner and outer diameters of the tubular body 12 vary depending on fluid pressure of fluid flowing through the graft 10. At low pressure, a maximum outer diameter OD (shown in FIG. 2A) of the graft 10 can be from about 3 mm to about 13 mm, and a maximum inner diameter ID (shown in FIG. 2A) of the graft 10 can be from about 2.5 mm to about 10 mm.

As shown in FIGS. 2A and 2B, the tubular body 14 and support frame 16 are configured such that as a pressure increases due to pulsating blood flow flowing through the lumen 14, the cross-sectional area of a portion of the lumen 14 increases, while a perimeter of the tubular body 12 remains substantially unchanged. As used herein, “substantially unchanged” may mean that the perimeter does not increase at all as fluid pressure increases or that any increase in perimeter of the tubular body 12 is minimal and/or is no more than would be expected for a conventional graft formed from ePTFE. As discussed previously, vascular grafts formed from substantially rigid materials, such as PTFE, generally do not expand radially outwardly by a substantial amount due to changes in physiologic fluid pressure.

In some examples, the tubular body 12 and support frame 16 are configured to transition between a first state (shown in FIG. 2A) in which the portion of the lumen 14 has a first cross-sectional area (shown by shape A1 in FIG. 2A) and a first circumference or perimeter (shown by oval C1 in FIG. 2A), and a second state (shown in FIG. 2B) in which the portion of the lumen 14 has a second cross-sectional area (shown by shape A2 in FIG. 2B) and a second circumference or perimeter (shown by circle C2 in FIG. 2B). The second cross-sectional area A2 is greater than the first cross-sectional area A1, and the second perimeter C2 is substantially equivalent in length to the first perimeter C1.

In some examples, the vascular graft 10 is configured to be responsive to changes in pressure that occur during pulsatile blood flow through vasculature of a patient. For example, the vascular graft 10 can be configured to be in the first state when pressure of fluid flowing through the lumen 14 is diastolic pressure for the patient. As is known in the art, normal diastolic blood pressure for a healthy human is from about 60 mmHg to about 80 mmHg. The graft 10 can be configured to transition to the second state as pressure of blood flowing through the lumen 14 increases above normal diastolic pressure towards normal systolic pressure for the patient. As is known in the art, systolic pressure for a healthy individual is from about 90 mmHg to about 120 mmHg. For individuals with systolic hypertension, the systolic blood pressure can be 130 mmHg or more. In some examples, the cross-sectional area A2 for the lumen 14 in the second state can be from about 5% to about 25%, or preferably about 10% to about 15%, greater than the cross-sectional area A1 in the first state. The dimension (i.e., length) for the perimeter C2 in the second state should be within at least about 5%, preferably within about 2% to about 3%, of the perimeter C1 in the first state. For example, the cross-sectional area A1 in the first state can be from about 2.0 mm² to about 600 mm² and the perimeter C1 can be from about 5 mm to about 105 mm. In the second state, the cross-sectional area A2 can be from about 3 mm² to about 900 mm² and the perimeter C2 can be from about 6 mm to about 105 mm. A maximum outer diameter OD of the tubular body 12 in the second state can be from about 3.0 mm to about 35 mm or greater than about 9 French. A maximum inner diameter ID of the tubular body 12 or diameter of the lumen 14 in the second state, defined by the inner surface 24 of the sidewall 22, can be from about 2 mm to about 33 mm.

As will be described in further detail hereinafter in connection with specific examples of the vascular graft 10, it is believed that a vascular graft 10 that transitions between the first state and the second state provides spring action in response to changes in fluid pressure, thereby allowing the tubular body 12 of the graft 10 to act essentially as a shock absorber, evening-out blood flow velocities, and absorbing pressure spikes. More specifically, the graft 10 can be configured such that as the internal pressure in the lumen 14 increases, the geometry of the tubular body 12 deforms from the first state to the second state, in which a geometry of the lumen 14 approaches a round cross-section or at least a rounder cross-section. The graft 10 can be configured to return to the original (e.g., non-round or oval) shape when the pressure is reduced. This allows for a somewhat radially compliant graft 10. However, in some instances, radial compliance may be limited by material creep and/or by formation of fibrin/scar tissue (external adhesions and/or internal intimal hyperplasia), limiting such flexibility.

Vascular Graft with Compressed Tubular Body

In some examples, the graft 10 comprises a tubular body 12 that has a round geometry in an unbiased state that is forced out-of-round into a biased state by the support frame 16, which acts as an external spring mechanism. For example, the support frame 16 can be configured to partially compress the tubular body 12, thereby providing a lumen 14 with a reduced cross-sectional area. As used herein, an “unbiased state” refers to a state were no external forces are applied either through the lumen 14 (e.g., forces from fluid flow and/or pressure) or to the exterior of the tubular body 12 (e.g., compressive or expanding forces from the support frame 16). As used herein, a “round geometry” refers to a lumen 14 having a cross-section that is substantially circular, such that, for example, a major diameter of the lumen 14 is within at least 5% of a length of each minor diameter of the lumen 14. Thus, a “round geometry,” in accordance with this definition includes circles, slightly oval shapes, slightly ovoid shapes, and other shapes falling within the scope of the above general definition. An “out-of-round” geometry refers to a lumen 14 having a cross-sectional shape that is irregular and/or a shape that is elongated or oblong, where a length of a major dimension is at least 5% greater than a length of at least one minor dimension. Thus, the “out-of-round” definition does not include circles; however, it does include oval shapes, ovoid shapes, and other shapes falling within the scope of the above general definition. The “out-of-round”shape can also be a shape with at least one straight side (e.g., a triangle, square, rectangle, polygon, trapezoid, etc.) or more than one straight side. The “out-of-round” shape can also be an irregular shape having sides of different lengths, such as an irregular polygon or crescent.

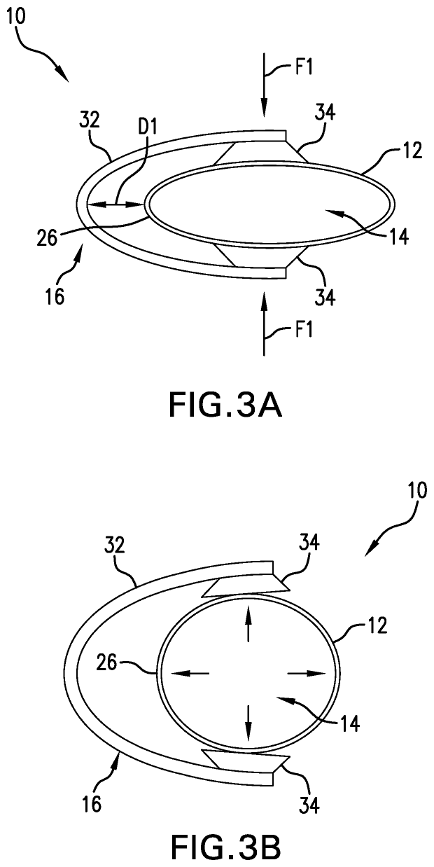

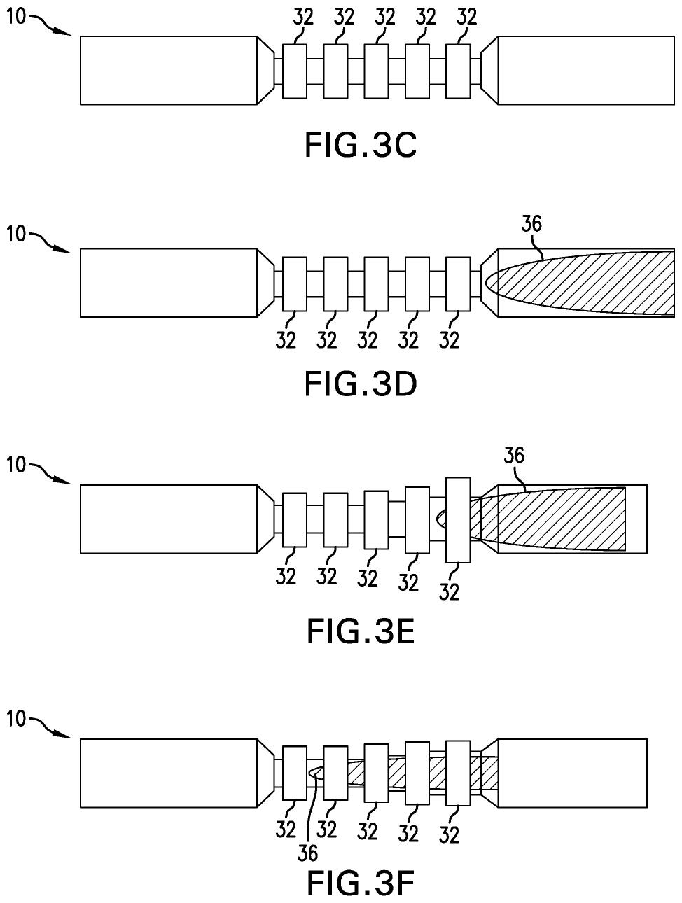

FIGS. 3A-3F show one example of a graft 10 comprising a tubular body 12 and a support frame 16 that compresses the tubular body 12 when no fluid is flowing through the lumen 14 and/or when fluid pressure is low. As described previously, the tubular body 12 is round in the unbiased (uncompressed) state, such as shown in FIG. 3B, and out-of-round in the biased state as shown in FIG. 3A. The support frame 16 is engaged to the tubular body 12 for modifying (e.g., compressing) the shape of the substantially round cross-section of the lumen 14 defined by the tubular body 12. In some examples, as shown in FIG. 3A, the shape of the cross-section of the tubular body 12 is an oval when the graft is in the first (e.g., low pressure) state.

The support frame 16 can comprise various structures configured to compress the tubular body 12 to create the oval shape. For example, the support frame 16 can comprise various combinations of bands, springs, clips, coils, and similar structures for compressing the tubular body 12 by exerting a radially inwardly directed pushing force F1 on the tubular body 12. In some examples, portions of the frame 16 can be formed from a material capable of being heat-set to adopt a shape at body temperature that compresses the tubular body 12. For example, portions of the support frame 16 can be formed from a metal alloy having shape memory and super-elastic properties, such as a nickel-titanium alloy (e.g., NITINOL). As is known in the art, shape memory materials, such as NITINOL, can be biased to a deployed position and/or can be configured to adopt the deployed position after being heated above a selected temperature, such as body temperature. Super-elastic materials are flexible and capable of transitioning between different shapes and configurations as external forces are applied to the support frame 16. Alternatively or additionally, portions of the support frame 16 can be formed from an elastomeric material.

As shown in FIGS. 3A-3F, in some examples, the support frame 16 comprises one or multiple pinching springs 32 placed in series along a portion of the tubular body 12. The pinching springs 32 can comprise mounting brackets 34 or anchors for contacting portions of the outer surface 26 of the tubular body 12, as shown in FIGS. 3A and 3B. It is believed that the pinching springs 32 act as a significant damper for fluid pressure spikes, as kinetic energy is redirected to the pinching springs 32. For example, as shown in FIG. 3D, a pressure surge or wave (illustrated as a shaded wave 36) of fluid can move towards the portion of the tubular body 12 compressed by the pinching springs 32. As the wave 36 contacts the compressed portion of the tubular body 12 (as shown in FIG. 3E), some springs 32 will move radially outwardly absorbing forces of the wave 36. For example, the first spring 32 in FIG. 3E is forced to an expanded state by the passing wave 36. As shown in FIG. 3F, as the wave 36 passes deeper through the graft 10, pressure of the dampened wave (shown by the unshaded wave 36 in FIG. 3F) is dissipated by multiple springs 32. After the wave 36 passes through the portion of the tubular body 12 compressed by the springs 32, the springs 32 return to the first or low pressure position (shown in FIG. 3C). In the first or low pressure position (FIG. 3C), all of the springs 32 compress the tubular body 12 into an oval shape as shown by FIG. 3A.

The pinching springs 32, or other structures forming support frames 16, can be connected to portions of the outer surface 26 of the tubular body 12 by any convenient mounting technique. For example, the pinching springs 32 or mounting brackets 34 can be fused to portions of the tubular body 12. In other examples, the support frame 16 can be connected to the tubular body 12 using various biocompatible adhesives, as are known in the art.

As shown in FIGS. 3A and 3B, the pinching springs 32 are connected to the outer surface 26 of the tubular body 12 at points on the minor diameter of the tubular body 12 (when the tubular body 12 is compressed to have the oval cross-section). The pinching springs 12 are spaced apart from the outer surface 26 of the tubular body 12 at other portions along the perimeter of the tubular body 12. For example, along a major diameter of the oval, a distance DI between the outer surface 26 of the tubular body 12 and an inner surface of the springs 32 can be from about 0.1 mm to about 3.0 mm. As used herein, “points on the minor diameter” for an oval refer to the points of the oval that intersect the shortest diameter or minor diameter of the oval, which is on the minor axis of the oval. In contrast, “points on the major diameter” for an oval refer to points of the oval that intersect the longest diameter of the oval, which is on the major axis of the oval.

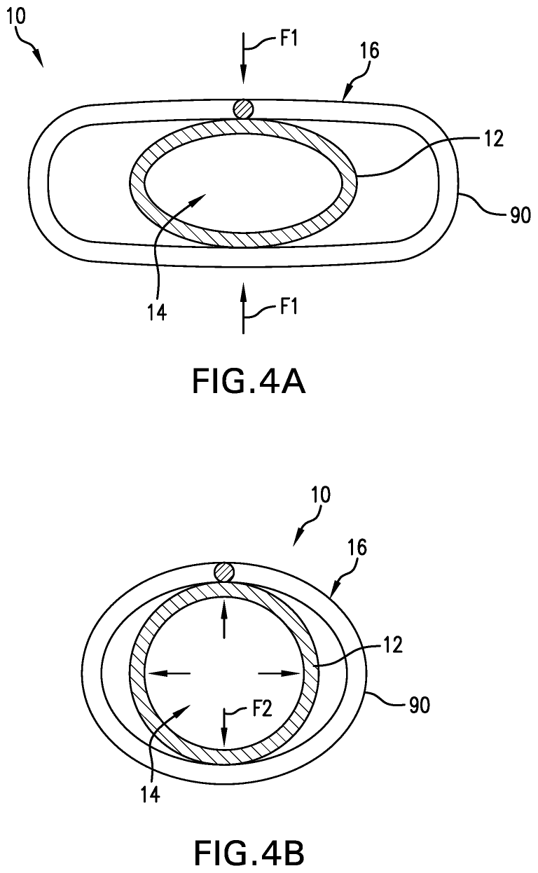

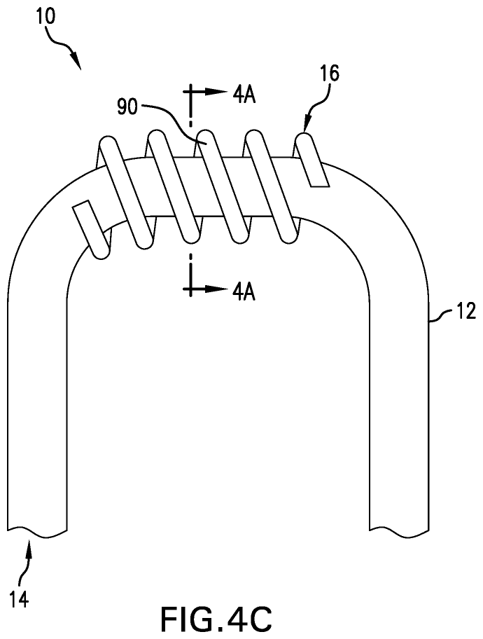

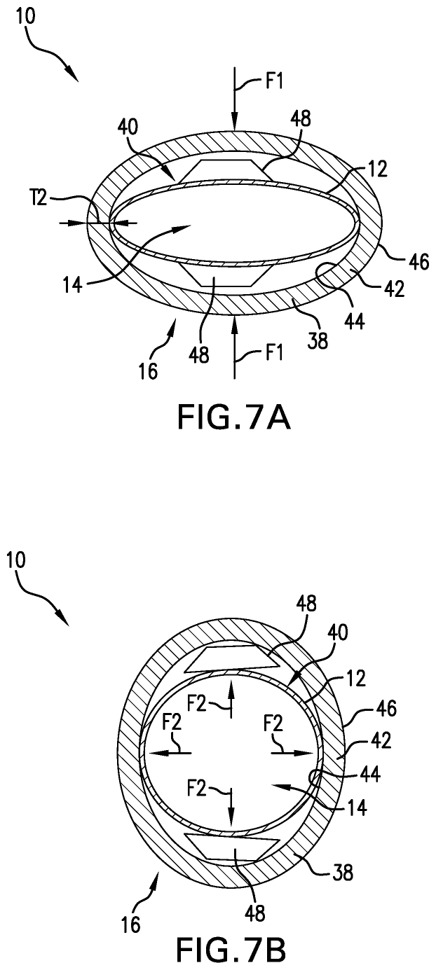

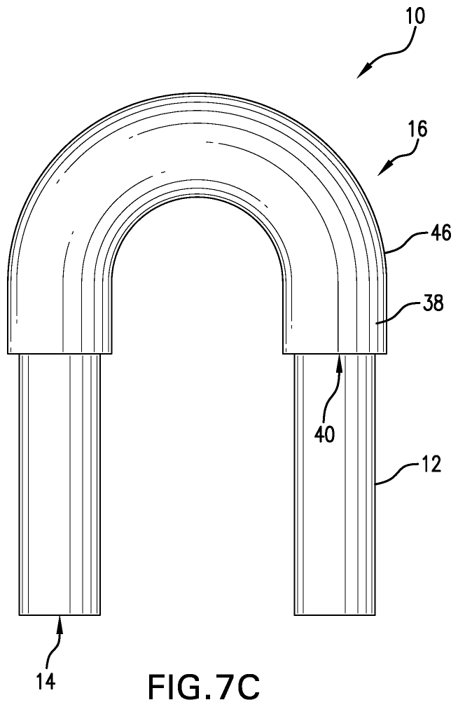

With reference to FIGS. 4A, 4B, and 4C, in some examples, the support frame 16 can comprise a coil 90, such as a helical coil, formed from an axially extending member. The support frame 16 may also be a sheath or sleeve as shown in FIGS. 7A, 7B, and 7C. As shown in FIGS. 4A, 4B, and 4C, the coil 90 can be an external coil mounted to an outer surface of the tubular body 12. In some examples, the support frame 16 could include two or more coils wound together to provide additional compression. In some examples, the coil(s) may comprise a PTFE coil/helix formed into an oval cross-section and adhered externally or internally to the round tubular body 12, or even formed within the sidewall 22 (shown in FIG. 1) of the tubular body 12. For example, the tubular body 12 can be adhered externally or internally to the coil 90 on the minor diameter of the coil 92 and tubular body 12.

In order to provide suitable compression for the tubular body 12, a lumen of the coil 90 is oval-shaped in an unbiased state. In such cases, the tubular body 12 can be adhered to the coil 90 on the minor diameter of both the oval-shaped coil 90 and the oval shaped tubular body 12. The oval shaped coil 90 compresses the round tubular body 12 in the same manner as the pinching springs 32 described previously, by exerting a compression force F1 (shown in FIG. 4A). As pressure builds within the tubular body 12 (shown by arrows F2), the tubular body 12 wants to become round. In order to become round, the tubular body 12 deforms the coil 90, causing the coil 90 to become more round. As the pressure reduces, the tubular body 12 and coil 90 return to the oval shape. This ability of the coil 90 to transition between a biased oval configuration and a round or rounder expanded configuration creates a pressure shock absorber effect to compensate for and absorb pulsatile fluid pressure in a similar manner to the previously described pinching springs 32.

### Methods of Forming a Vascular Graft

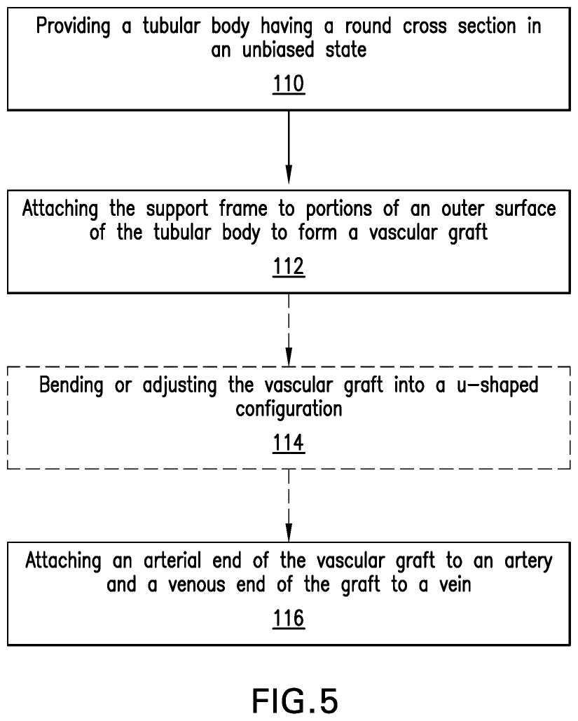

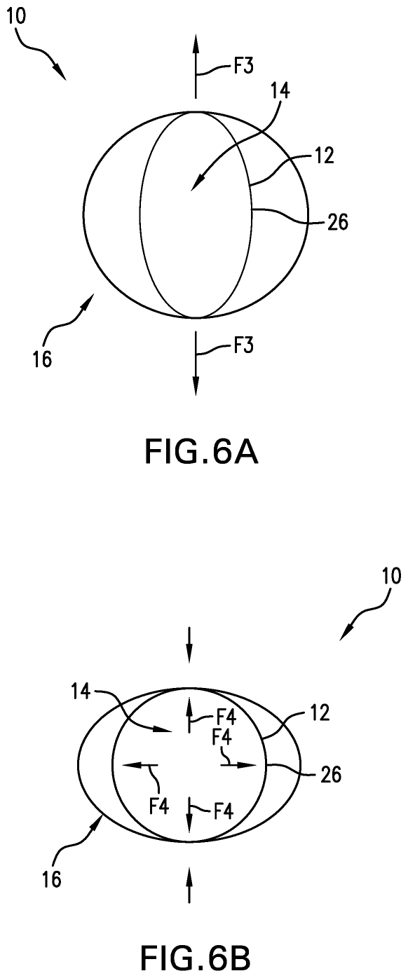

A flow chart showing steps of a method for forming the vascular graft is shown in FIG. 5. At step 110, the method includes providing a tubular body 12 defining a lumen 14. As previously described, in an unbiased state, the tubular body 12 can have a round cross-section that becomes oval, oblong, or out-of-round when compressed by the support frame 16. In other examples, as described in connection with FIGS. 6A and 6B, the tubular body 12 can be oval shaped in the unbiased state. In either case, the tubular body 12 can be formed by an extrusion process as known in the art, or by any other suitable process for forming implantable tubular members comprising biocompatible materials.

At step 112, the method also includes attaching the support frame 16 to the tubular body 12 at one or more portions of the tubular body 12. As previously described, the support frame 16 is configured to modify a shape of a cross-section of at least a portion of the lumen 14. For example, the support frame 16 can be configured to compress the tubular body 12 thereby causing the cross-section of the lumen 14 to become oblong, oval shaped, or out-of-round. The support frame 16 can extend along substantially the entire length of the tubular body 12. In other examples, the support frame 16 may only engage a portion of the tubular body 12 or only engage multiple discrete portions of the tubular body 12. For example, the support frame 16 may be positioned on the u-bend segment 30 of the vascular graft 10, as shown in FIG. 1.

As in previous examples, once connected together, the tubular body 12 and support frame 16 are configured so that as a pressure and/or flow rate of fluid flowing through the lumen 14 increases due to pulsatile flow a cross-sectional area of the lumen 14 increases, while the perimeter of the portion of the lumen 14 remains substantially unchanged. For example, the vascular graft 10 can be configured such that, when the support frame 16 is attached to the tubular body 12, the tubular body 12 and support frame 16 conform to a first state (i.e., a biased state or out-of-round state) when pressure of fluid flowing through the lumen 14 is diastolic pressure and conform to a second state (i.e., an expanded state or round or more round state) when pressure of the fluid flowing through the lumen 14 increases to systolic pressure. For example, the vascular graft 10 can be configured to be in the first state when pressure of blood flowing through the lumen 14 is less than 90 mmHg or from about 45 mmHg to about 90 mmHg. The vascular graft 10 can be configured to begin to transition to the second state when the pressure of fluid through the lumen 14 increases, becoming greater than or equal to about 90 mmHg. Also as previously discussed, the cross-sectional area of the lumen 14 in the second state can be from about 5% to about 20%, or preferably about 10% to about 15%, greater than the cross-sectional area of the lumen 14 when the vascular graft 10 is in the first state.

The support frame 16 can comprise any of the previously described structures for compressing the round lumen 14 of the tubular body 12, including, for example, one or more bands, springs, clips, clamps, coils, tines, sheaths, sleeves, and other members. The support frame 16 can be connected to portions of the tubular body 12 by any convenient technique including by fusing or sintering portions of the tubular body 12 to the support frame 16 and/or by using a biocompatible adhesive. When fusing or sintering components of the graft 10 together, it may be desirable that the materials used to form the respective components have a softening or sintering temperature that permits their bonding without damage to their structural integrity. For example, the support frame 16 and tubular body 12 may be made of the same material, such as ePTFE. As previously described, in some examples, the support frame 16 is only connected to points along the minor diameter of the tubular body 12, as shown in FIGS. 3A and 4A. Other portions of the support frame 16 are spaced apart from the tubular body 12. Further, as previously described, in some examples, the support frame 16 is provided along an entire length of the tubular body 12. In other examples, the support frame 16 is provided along certain segments of the tubular body 12, while other segments of the tubular body 12 do not include structures for modifying the cross-sectional area of the lumen 14 of the tubular body 12.

At step 114, once the tubular body 12 and the support frame 16 are connected together, optionally, the vascular graft 10 can be bent or adjusted to fit within a desired surgical or implant site. For example, the vascular graft 10 can be bent, as shown in FIG. 1, to include substantially parallel straight segments 28 and the u-bend segment 30 between the straight segments 28. In other examples, the vascular graft 10 can be bent or adjusted into any other convenient configuration depending on the desired deployment location and intended use. In other examples, the vascular graft 10 may not be bent to the u-shaped configuration. Instead, the vascular graft 10 may be deployed in a substantially straight (e.g., unbent) configuration.

At step 116, the method can further include attaching the ends 18, 20 of the tubular body 12 to body structures to establish fluid flow through the tubular body 12. For example, a first end or arterial end 18 of the tubular body 12 can be connected to an artery using any convenient surgical technique for creating an anastomosis between an artery and the graft 10. Creation of the anastomosis can include, for example, making an incision in a sidewall of the artery and securing the first or arterial end 18 to the artery surrounding the incision using conventional techniques, such as suturing. In a similar manner, the second end or venous end 20 of the tubular body 12 can be secured to a vein by forming an incision in a sidewall of the vein and attaching the end of the tubular body 12 to the vein about the incision. When the ends 18, 20 of the tubular body 12 are connected to the artery and vein, the graft 10 forms an arterio-venous shunt for transporting pulsating blood from the artery to the vein through the graft 10. The graft 10 or shunt can be used for dialysis procedures, as previously described. During such procedures, the tubular body 12 can be punctured by needles to access the lumen 14 of the tubular body 12.

Vascular Graft with Expanded Tubular Body

With reference to FIGS. 6A and 6B, in other examples, the vascular graft 10 comprises a support frame 16 biased to cause portions of a tubular body 12 to expand radially outwardly rather than compressing portions of the tubular body 12, as in the previous examples. In such examples, the implantable tubular body 12 can be either round, oval-shaped, or out-of-round in an unbiased state. The support frame 16 engages the tubular body 12 and exerts a radially outwardly directed pulling force (shown by arrows F3) on the tubular body 12 causing a cross-section of a lumen 14 of the tubular body 12 to become oblong or oval-shaped, as shown in FIG. 6A.

As in previous examples, the support frame 16 can comprise resilient spring-like structures configured to engage the outer surface 26 of the tubular body 12 at selected points on the outer surface 26 and to pull the connection points away from each other, thereby causing the shape of the cross-section of the lumen 14 to become more elongated or oblong. The support frame 16 that expands the tubular body 12 can comprise, for example, bands, springs, slips, helical coils, and similar resilient structures configured to radially expand portions of the tubular body 12 when pressure and/or flow rate of fluid through the lumen 14 is low. For example, the support frame 16 can comprise a shape-memory material, such as NITINOL, heat-set or biased to expand portions of the tubular body 12 radially outwardly relative to other portions of the tubular body 12. The NITINOL support frame 16 can be disposed on an inner surface 24 of the tubular body 12 or can be embedded within the sidewall 22 of the tubular body 12.

As the fluid pressure and/or flow rate increases, the increased pressure exerts an outwardly directed force (shown by arrows F4 in FIG. 6B) on the tubular body 12 that overcomes the expanding pulling force of the support frame 16, causing the other portions of the tubular body 12 to move radially outwardly, thereby causing the shape of the cross-section of the tubular body 12 to become more round, as shown in FIG. 6B, while causing the support frame 16 to compress.

In some examples, the expanding support frame 16 comprises a sheath formed from a member extending axially over the outer surface 26 of the tubular body 12. In some examples, the expanding support frame 16 comprises a helical coil formed from members or tines extending axially over the outer surface 26 of the tubular body 12. In some examples, the support frame 16 may include two or more coils wound together to provide a greater expanding force. The central lumen defined by the coil(s) can have a round cross-section in an unbiased state. For example, the coil(s) can be a metallic coil(s) formed from a shape memory alloy (e.g., NITINOL) that are configured to define a round lumen at body temperature. The coil(s) can be mounted to the outer surface 26 of the tubular body 12 along the major dimension of the oval-shaped tubular body 12, as shown in FIG. 6A. In other examples, as previously described, coils of the support frame 16 can be mounted to the inner surface 24 of the tubular body 12. Since the coil(s) is biased to define the round lumen, the coil(s) pulls connection points with the tubular body 12 away from each other causing the lumen 14 of the tubular body 12 to have the oval cross-section, shown in FIG. 6A. As the fluid pressure increases, other portions of the tubular body 12 bulge radially outwardly, overcoming the expanding force of the coil(s). In this second or high pressure state, the increased fluid pressure causes the coil(s) to become non-round and the cross-section of the lumen 14 of the tubular body 12 to become rounder or round, as shown in FIG. 6B. As the pressure reduces, the cross-section of the lumen 14 of the tubular body 12 returns to an oval shape and the coil(s) of the support frame 16 become round (as shown in FIG. 6A). Accordingly, the tubular body 12 and support frame 16 create a pressure shock absorber effect as the graft 10 and coil(s) transition between the low pressure and high pressure states.

### Vascular Graft and Elastomeric Sheath or Sleeve

With reference to FIGS. 7A, 7B, and 7C, in some examples, the vascular graft 10 comprises the tubular body 12 having a round lumen 14 in an unbiased state and a support frame 16 comprising an external sheath or sleeve 38. Similar to the coil(s) in previous examples, the sleeve 38 is configured to provide a spring force for modifying a shape of the cross-section of the lumen 14 of the tubular body 12 so that is it out-of-round when fluid pressure in the lumen 14 is low. The sleeve 38 can be formed from any convenient biocompatible material, such as PTFE, and/or from biocompatible elastomeric materials, such as silicone or synthetic rubber. The sleeve 38 can be configured to absorb forces exerted on the sleeve 38 as high pressure fluid passes through the lumen 14 of the tubular body 12. The sleeve 38 desirably is sufficiently flexible to transition from a low pressure state (shown in FIGS. 7A and 7C), where a lumen 40 of the sleeve 38 has an oval cross-section, for example, to a high pressure state (shown in FIG. 7B), where the lumen 40 of the sleeve 38 becomes rounder or round.

In some examples, as shown in FIGS. 7A and 7B, the elastomeric sleeve 38 comprises a sidewall 42 having an inner surface 44 and an outer surface 46. The sidewall 42 can have a thickness T2 of about 0.1 mm to about 2.0 mm in an unbiased or relaxed state. The thickness of the sidewall 42 can change as pressure within the lumen 14 increases from the low pressure state to the high pressure state. The amount the thickness changes is dependent upon elastomeric properties (i.e., durometer and stretch properties) of materials that form the sleeve 38. In some examples, the sleeve 38 also comprises inner extension portions or spacers 48 connected between the inner surface 44 of the sidewall 42 and the outer surface 26 of the tubular body 12. The sleeve 38 and/or spacers 48 can be connected to the outer surface 26 of the tubular body 12 by known processing techniques, such as adhesive bonding, solvent bonding, hot-melt bonding, or sintering, as are known in the art.

The sidewall 42 and spacers 48 (if present) can define the oval lumen 40 in the low pressure state, as shown in FIG. 7A. The sleeve 38 is configured to exert a compressive force (shown by arrows F1 in FIG. 7A) on opposing sides of the tubular body 12, causing the opposing sides to compress, such that the shape of the cross-section of the lumen 14 of the tubular body 12 becomes non-round, oval-shaped, or out-of-round. As pressure for fluid in the lumen 14 increases (as shown in FIG. 7B), the compression force from the sleeve 38 is overcome, causing portions of the lumen 14 of the tubular body 12 to bulge radially outwardly (shown by arrows F2 in FIG. 7B) and the elastomeric sleeve 38 to become rounder or round. When the fluid pressure reduces, the sleeve 38 returns to the oval shape, which compresses the tubular body 12, as previously described. In this way, the elastomeric sleeve 38 serves as a pressure shock absorber that compensates for and absorbs pulsatile fluid pressure in a similar manner to the previously described pinching springs 32 and external sheath 90.

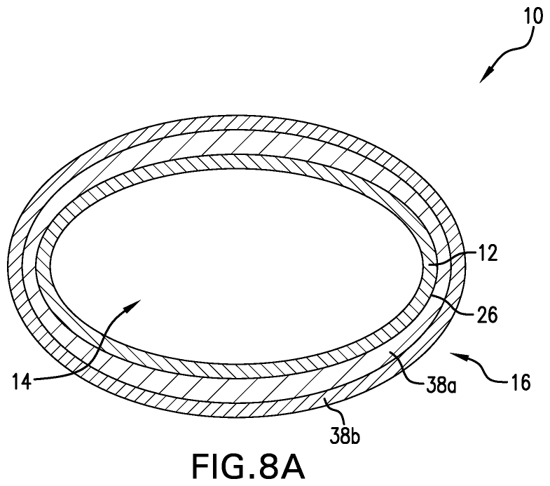

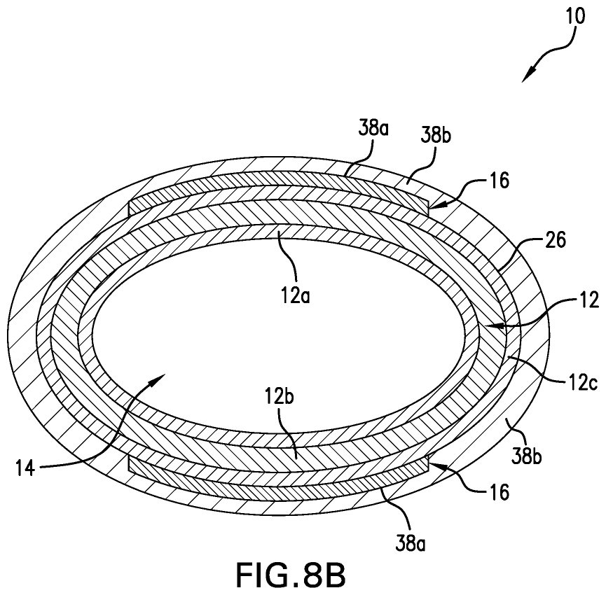

In other examples, as shown in FIGS. 8A and 8B, the tubular body 12 and support frame 16 comprising an elastomeric sleeve (comprising layers 38a, 38b) can be integrated into a unitary or integral multi-layer tubular structure. For example, the tubular body 12 can comprise an extruded single-layer tube formed from a suitable biocompatible material, such as ePTFE. An exemplary graft comprising a single-layer tubular body 12 that can be adapted to include the dampener of the present disclosure is the EXXCEL™ Soft ePTFE vascular graft by Maquet Cardiovascular, LLC. The graft 10 may also comprise a tubular body 12 comprising multiple layers, such as layers 12a, 12b, 12c, laminated together as shown in FIG. 8B. An exemplary multi-layer graft that can be adapted to include the dampener of the present disclosure is described in U.S. Patent Application Publication No. 2018/0345624 A1, which is incorporated herein by reference for all it discloses.

With continued reference to FIGS. 8A and 8B, the support frame 16 can be connected to an outer surface 26 of the single layer (FIG. 8A) or multilayer (FIG. 8B) tubular body 12. The support frame 16 comprises one or more layers 38a, 38b of resilient and/or elastomeric material connected or adhered to the outer surface 26 of the tubular body 12 so as to compress the tubular body 12 into an oval, as shown in FIGS. 8A and 8B, or other other-than-round configuration. For example, the layer or layers 38a, 38b of support frame 16 can be laminated to the outer surface 26 of one or more layers that form the tubular body 12, thereby forming a vascular graft 10 comprising a multi-layer tubular structure provided with pressure shock absorbing functionality. In some examples, the layers 38a, 38b can be laminated around the entire perimeter of the tubular body 12 (as shown in FIG. 8A) and/or along an entire length of the tubular body. In other examples, the layers 38a, 38b may only be applied to particular portions of the tubular body 12, such as on the minor dimensions of the oval shaped tubular body 12, as shown in FIG. 8B.

In some examples, the graft 10 can be configured such that in an unbiased state the lumen 14 of the tubular body 12 has an elongated or oval shaped cross-section. As a pressure of fluid flowing through the lumen 14 increases, the cross-sectional area of the lumen 14 increases because the increased pressure forces the lumen 14 into a rounder or round configuration, while a perimeter of the lumen 14 remains substantially unchanged. For example, the shape of the cross-section of the lumen 14 can be round in the high pressure or second state, as in previous examples. The lumen 14 can be configured to return to the unbiased state when the pressure of fluid flowing through the lumen 14 decreases and/or when no fluid is flowing through the lumen 14. In other words, the tubular structure of the graft 10 may be biased to assume an oval, other non-round, or out-of-round configuration for itself and the lumen 14 that reconfigures during pulsatile increases in fluid pressure so as to assume a rounder or round configuration in order to provide a pressure shock absorbing effect.

Vascular Graft with Manufactured Out-of-Round Tubular Body

In some examples, as described in further detail herein in connection with FIGS. 9A-9D and 10A-10C, a vascular graft 210 or tubular body 312 is made to have inherent flexibility and resiliency, such that the tubular body 212, 312 can transition between a first or low pressure state and a second or high pressure state without requiring a separate support frame to compress or expand the tubular body, as in previous examples. For example, the vascular graft 210 can be intentionally manufactured out-of-round or in a variety of patterned geometries selected and configured to provide a structure with variable volume. As previously described, an “out-of-round” geometry refers to a lumen 214 having a cross-sectional shape that is irregular and/or a shape that is elongated or oblong, where a length of a major dimension is at least 5% greater than a length of at least one minor dimension. The “out-of-round” shape can also be a shape with at least one straight side (e.g., a triangle, square, rectangle, polygon, trapezoid, etc.) or more than one straight side. The “out-of-round” shape can also be an irregular shape having sides of different lengths, such as an irregular polygon or crescent.

As described in further detail hereinafter, as the internal pressure for fluid flowing through the graft 210 increases, the geometry deforms from the initial geometry (e.g., the first state) to approach a rounder or round cross-section (e.g., the second state). As used herein, “approaches a rounder or round cross-section” or “approaching a rounder or round cross-section” means that the cross-section transforms to a shape that is closer to circular than the initial shape of the first state. However, the shape does not necessarily become a circle. Instead, approaching rounder or round may refer to a transformation in which the difference between the length of the major dimension and the length of the minor dimension decreases by an appreciable amount. In some examples, a diameter of the graft 210 may decrease by about 30% to about 50% as the graft transitions between the fully pressurized state (e.g., the rounder or round second state) and the unpressurized state (e.g., the initial oblong or first state).

In other examples, a shape may approach a rounder or round configuration when straight portions of the shape bulge outwardly becoming curved, such that the perimeter of the shape becomes more circular, as occurs when a square transforms to a circle. As in previous examples, the tubular body 212 may also be configured to return to the original or initial shape when pressure of fluid flowing through the lumen 214 of the tubular body 212 decreases and/or when no fluid is flowing through the lumen 214. As in previous examples, it is believed that this radial compliance and flexibility can replicate, either more so or less so, a natural vessel's ability to change volumetric carry capacity, thereby providing a radially compliant graft 210.

The lumen 214 of the tubular body 212 can be any shape capable of transitioning between the first state and the second state without substantially changing the perimeter of the tubular body 212. In other words, the shape of the tubular body 212 may assume different out-of-round, rounder, and/or round configurations; however, the sidewall of the tubular body 212 does not stretch radially. Accordingly, the shape of the cross-section of the sidewall of the tubular body 212 and lumen 214 substantially contributes to the performance of the tubular body 212. The tubular body 212 is not intended to be particularly radially compliant or stretchable and, for example, the tubular body 212 does not change shape by stretching portions of the tubular body 212 radially outwardly, which would increase the circumference or perimeter of the tubular body 212. Instead, the tubular body 212 is formed such that, in the unbiased state or first state, portions of the sidewall are folded, recessed, or oblong. As pressure increases, the portions of the sidewall begin to unfold causing the lumen 214 of the tubular body 212 to become round or rounder. Of course, it should be remembered that the tubular body 212 may be axially compliant or stretchable so as to be able to bend more or less as does the tubular body 12 shown in FIG. 1.

The tubular body 212 can be formed by any conventional method for forming tubular polymeric structures such as, for example, by extrusion. An example is an extruded star-shaped pattern or a triangle or trapezoid. In order to achieve these complex, non-round shapes, a die insert and mandrel of an extrusion machine would need to be shaped and aligned to each other during the extrusion process. In other examples, complex cross-sectional shapes can be obtained by forced reshaping post-extrusion and/or post sintering to form a cross-section in such a non-round shape.

FIG. 9A shows vascular grafts 210 with different cross-sectional shapes in the first or low pressure state, where the shape is “out-of-round”. Various non-limiting cross-sectional initial out-of-round shapes illustrated in FIG. 9A include a triangle shape, a crescent shape, various star shapes (i.e., four point, five point, and seven point), a diamond shape, a rectangular shape with rounded, blunted, or truncated corners (i.e., an elongated pill shape), a trapezoid shape, or an oval shape. As used herein, an “elongated pill shape” can refer to an elongated shape having curved portions or curved corners, such as an oval or a rectangle having curved corners as shown in FIG. 9A. FIG. 9B shows the cross-sectional shapes in the second or high pressure state where the shape becomes rounder and/or “approaches round.” The corresponding non-limiting expanded shapes illustrated in FIG. 9B include a circle shape (corresponding to the previous triangle shape), a concave shape (corresponding to the previous crescent shape), various expanded star shapes corresponding to the previous four point star shape, five point star shape, and seven point star shape, respectively, an oval shape (corresponding to the previous diamond shape), a nearly oval shape (corresponding to the previous rectangular shape with rounded corners), and a nearly round shape (corresponding to the previous trapezoid shape). The shapes in FIGS. 9A and 9B are not necessarily drawn to scale and may show a larger volume change than is actually needed for the graft 210 disclosed herein. As previously described, the volume change between the first or low pressure state and the second or high state can be about 5% to 20% or preferably from about 10% to about 15%. Further, the shapes shown in FIGS. 9A and 9B are not intended as limiting and any other out-of-round shape that can approach round as fluid pressure increases may be used within the scope of the present disclosure.

As shown in FIGS. 9A and 9B, the cross-sectional shape of the grafts 210 can be, for example, an oval, ellipse, triangle, rectangle, or another regular or irregular polygon. In other examples, the “out-of-round” shape can be a regular or irregular shape having recesses, recessed portions, cutaway portions, or similar irregularities. For example, the shape can be a star having three or more points. While any non-round cross-sectional shape can be used within the scope of the present disclosure, some shapes allow for greater volume expansion than others. However, it is believed that the more complex shapes may be less resistant to kink. Therefore, in some examples, a helical coil could be applied to portions of the tubular body 212 to provide added kink resistance. In some examples, the out-of-round or complex geometry can span the entire length of the graft 210. In other examples, the out-of-round or complex geometry may span only a portion of the length of the tubular body 212 and other portions of the tubular body 212 may be round.

In some examples, as described in further detail herein in connection with FIGS. 9C and 9D, a vascular graft is made to have inherent flexibility and resiliency as a result of a non-concentric variable wall thickness, such that the tubular body 312 can transition between a first or low pressure state (FIG. 9C) and a second or high pressure state (FIG. 9D) without requiring a separate support frame to compress or expand the tubular body, as in previous examples, because a thin portion 317 of the wall 315 is configured to pulse its diameter by moving radially outwardly as pressure increases. More specifically, in this case, the thinner portion 317 of the wall 315 serves to form a pivot point 320 at the intersection of the thin portion 317 and the thicker portion 321 of the wall 315. Pulsation dampening is achieved based on the substantial difference between diastolic and systolic pressures causing the tubular body 312 to adjust its configuration between a relaxed state (shown in FIG. 9C) corresponding to when blood pressure is lower, such as when pressure is diastolic or thereabouts, and a flexed state (shown in FIG. 9D) corresponding to when pressure in the tubular body 312 is substantially higher, such as occurs closer to and around systolic pressures. FIG. 9C shows the cross-section of such a non-limiting tubular body configuration provided with a non-concentric variable wall thickness in which the tubular body 312 is in the relaxed state when exposed to diastolic pressures. Phantom line 319 in FIG. 9C illustrates the contour that thin portion 317 may flex to when pressures within the tubular body 312 are high enough to flip the configuration of the tubular body from the relaxed state of FIG. 9C to the flexed, expanded state of FIG. 9D. Thus, FIG. 9D shows the same tubular body 312 of FIG. 9C when pressures inside the tubular body 312 have risen high enough during pulsatile blood flow to flip the tubular body to the flexed state of FIG. 9D.

In accordance with some aspects of this disclosure, the thin portion 317 possesses about 10-90% of the thickness of the thicker portion 321 of the wall 315. In accordance with some aspects of this disclosure, the thin portion 317 possesses about 20-80% of the thickness of the thicker portion 321 or about 10-50% of the thickness of the thicker portion 321. The cross-sectional configuration of the relaxed state of tubular body 312 of FIG. 9C is illustrated as a crescent moon shape when the tubular body 312 is subjected to lower pressures within its lumen and the cross-sectional configuration of the flexed state of the tubular body 312, as shown in FIG. 9D, is round or almost round when pressures within the lumen of tubular body 312 are sufficiently high to force the tubular body 312 to transition from the relaxed state to the flexed state. Likewise, when the tubular body 312 is in the flexed state, as pressures within the lumen fall below a threshold pressure, then the elastic forces of the wall 315 of the tubular body 312 return automatically to the relaxed state of FIG. 9C. In other words, elastic forces of the wall 315 bias the wall 315 to the relaxed state, whereas rising fluid pressures within the lumen of the tubular body 312 during pulsatile blood flow can overcome the biasing elastic forces of the wall 315 of the tubular body 312, thereby causing it to flip to the flexed state of FIG. 9D when internal pressure within the lumen of the tubular body 312 exceeds a threshold pressure.

While FIG. 9C illustrates an non-limiting embodiment of the relaxed state in which the cross-sectional configuration of the tubular body 312 has a crescent moon shape, in accordance with other non-limiting embodiments the tubular body 312 provided with a non-concentric variable wall thickness may be provided with some other configuration, such as those shown in FIGS. 9A and 9B, in which only a portion of the wall is thin and the rest of the wall is thicker. In other non-limiting embodiments, multiple portions of the wall may be thin separated by portions of the wall that are thicker.

Vascular Graft that Transitions to an Out-of-Round Shape

With reference to FIGS. 9E-9H, in some examples, a vascular graft 410 can also be configured to transition from a round or rounder configuration, in a relaxed or low pressure state, to an out-of-round configuration, in a high pressure state, to provide pulsation dampening. As in the examples of FIGS. 9A-9D, the vascular graft 410 is made to have inherent flexibility and resiliency, such that a tubular body 412 of the graft 410 can transition between the low pressure state and the high pressure state without requiring a separate support frame to compress or expand the tubular body. Unlike in previous examples, the vascular graft 410 is manufactured in the rounder or substantially round configuration and is configured to transition to the out-of-round configuration as pressure increases.

For example, as shown in FIGS. 9E-9H, the vascular graft 410 comprises a tubular body 412 with a non-concentric variable wall thickness that allows the graft 410 to transition from a first or low pressure state (FIGS. 9E and 9G) to a second or high pressure state (FIGS. 9F and 9H). As in previous examples, the tubular body 412 includes thin portion(s) 417 of a wall 415 of the tubular body 412 and thicker portion(s) 421. As in previous examples, the thinner portion(s) 417 of the wall 415 expand radially outward as pressure increases within the lumen of the tubular body 412, which changes a shape of the cross-sectional area of the tubular body 412. The changing of shape of the cross-section of the tubular body 412 absorbs or dampens pulsation of blood flow passing through the tubular body 412, in a manner similar to dampening provided by previously described examples.

FIGS. 9E and 9F show a cross-sectional view of a vascular graft 410 including a thin portion 417 on one side of the tubular body 412 and a thicker portion 421 on an opposing side of the tubular body 412. In FIG. 9E, which shows the relaxed state, the graft 410 has a round or nearly round cross-sectional shape. In the high pressure state, the thinner portion 417 deforms or bulges radially outwardly, as shown by arrow F3. In the high pressure state, shown in FIG. 9F, the tubular body 412 has an asymmetrical out-of-round oval, elliptical, oblong or egg shape.

FIGS. 9G and 9H show a cross-sectional view of another example of a vascular graft 410 including a symmetrical arrangement of thinner portions 417 and thicker portions 421. More specifically, the tubular body 412 of the vascular graft 410 includes two thinner portions 417 on opposing sides of the tubular body 412. The tubular body 412 also includes two thicker portions 421 on opposing sides of the tubular body 412 so that there is a thin portion 417 alternating with a thicker portion 421 when traversing the circumference of the tubular body 412. For example, as shown in FIGS. 9G and 9H, the thinner portions 417 are positioned on right and left sides of the tubular body 412. Thicker portions 421 are positioned on top and bottom sides of the tubular body 412. As shown in FIG. 9G, in the relaxed state, the tubular body 412 has a substantially round or rounder configuration. As shown in FIG. 9H, as pressure increases, the thinner portions 417 of the tubular body 412 move or bulge radially outwardly in a direction of arrows F4, causing the tubular body 412 to transition to an out-of-round configuration. While the embodiment illustrated in FIGS. 9G and 9H includes two thin portions 417 and two thicker portions 421, it is within the scope of this disclosure to form tubular bodies 412 possessing three thin portions 417 alternating with three thicker portions 421, or four thin portions 417 alternating with four thicker portions 421, and so on.

Vascular Graft with Spiral Flow

With reference to FIGS. 10A, 10B, and 10C, in some examples, the vascular graft 210 is configured to create a spiral fluid flow through the tubular body 212. Creation of a spiral fluid flow is believed to improve efficiency of blood flow through the lumen 214 because spiral laminar blood flow is understood to be the predominant type of arterial blood flow as reported by P. A. Stonebridge et al., Spiral Laminar Flow: a Survey of a Three-Dimensional Arterial Flow Pattern in a Group of Volunteers, 52 EUR. J. VASC. ENDOVASC. SURG. 674-680 (2016).

As shown in FIGS. 10A, 10B, and 10C, the vascular graft 210 can comprise the implantable tubular body 212 defining the lumen 214. As in previous examples, the tubular body 212 is configured to transition from a first state (shown in FIGS. 10A and 10B) in which at least a portion of the lumen 214 has a first cross-sectional area and a first perimeter, and a second state (shown in FIG. 10C) in which at least a portion of the lumen 214 has a second cross-sectional area and a second perimeter. The second cross-sectional area is greater than the first cross-sectional area and the second perimeter is substantially equivalent to the first perimeter.

In the first state, the tubular body 212 is star-shaped having raised ridges 250 forming points of the star that extend axially along a length of the tubular body 212 (as shown in FIG. 10A) and recessed portions 252 positioned between the raised ridges 250. The star shaped tubular body in FIGS. 10A-10C is a four-point star having four raised ridges 250 and four recessed portions 252. However, this configuration is not meant to be limiting, and in other examples, the star shaped cross-section may have three points or may have more than four points and a corresponding number of recessed portions. In this context, the term “point” or “points” should be construed to include tips that are smoothly rounded, blunted, or truncated as well as tips converging to a sharper edge. This definition pertaining to the term “point” or “points” applies to both the external “points” of the tip of each arm of a star-shaped tubular body and to the internal “points” of each recessed portion that exists between adjacent arms and form protrusions into the lumen of the star-shaped tubular body. Such smoothly rounded, blunted or truncated points may avoid certain impediments to blood flow in the lumen of the tubular body that could otherwise occur with sharper points. In other examples, the tubular body 212 may include only one raised ridge 250.

The raised ridges 250 define channels 254 (shown in FIG. 10B) of the lumen 214 that create or define a flow path for fluid flowing through the lumen 214 of the graft 210. The raised ridges 250 spiral about a central longitudinal axis of the tubular body 212, which creates spiraling or twisting flow paths through the lumen 214. In some examples, the flow paths defined by each of the multiple channels 254 can complete about one full rotation about the perimeter of the tubular body 212 over an axial length L2 (shown in FIG. 10A) of the tubular body 212. In other examples, the flow path(s) may complete multiple rotations about the perimeter of the tubular body 212 over the axial length L2 of the tubular body 212.

As shown in FIG. 10C, which shows the graft 210 in the second or high pressure state, as fluid pressure increases in the lumen 214, the recessed portions 252 move radially outwardly in a direction of arrow F2 from the central axis of the tubular body 212 so that the lumen 214 of the tubular body 212 becomes rounder or approaches round.

A tubular body 212 having a star-shaped cross-section that spirals or twists along a length of the tubular body 212 can be formed by conventional techniques for forming plastic tubing. For example, the tubular body 212 can be extruded using processes known in the art. In particular, a die of an extrusion machine can be rotated slowly during extrusion to create the spiraling raised ridges 250. The spiraling raised ridges 250 can also be created by post-processing techniques, as are known in the art.

In some examples, the tubular body 212 can be formed to include alternating compliant and less compliant regions to allow the tubular body 212 to transition from the first or low pressure state to the high pressure state. For example, the recessed portions 252 of the star-shaped cross-section can be formed from flexible or compliant materials so that the recessed portions 252 can move radially outwardly as pressure increases, causing the shape of the cross-section of the tubular body 212 to become rounder or approach round. In other examples, a thickness of the sidewall 226 of the tubular body 212 may vary to provide radial compliance. For example, the recessed portions 252 of the sidewall 226 may be thinner than the raised ridges 250 of the sidewall 226 that form points for the star-shaped cross-section. The thinner recessed portions 252 can be configured to move radially outwardly as pressure of fluid flowing through the tubular body 212 increases, as previously described.

Alternatively or in addition, the vascular graft 210 can include one or more of the previously described support frames for modifying a shape of the cross-section of the lumen 214, to cause the tubular body 212 to have the star-shaped cross-section when fluid pressure is low. For example, the support frame can include tines or other metallic members that compress portions of the tubular body 212 to form recessed portions 252. Portions of the tubular body 212 that are not contacted and/or compressed by the tines or other members can form the raised ridges 250 or points of the star-shaped cross-section. As previously described, as fluid pressure for fluid in the lumen 214 of the tubular body 212 increases, the increasing pressure overcomes the compression force applied by the support frame, which causes the recessed portions 252 of the tubular body 212 to move radially outwardly providing the lumen 214 with a rounder cross-section, as shown in FIG. 10C.

In accordance with this disclosure, the pressure shock absorber effect of the various disclosed embodiments of the support frame, whether constructed as a single integrated component or as an assembly, compensates for and absorbs pulsatile fluid pressure in the lumen of the tubular body. The compensation or absorbance of the pulsatile fluid pressure may be complete, such as evident from FIGS. 3D to 3F, or partial, such that some attenuated residual pulsatile pressure remains in the fluid flow. Thus, in some embodiments, the assembled components of the support frame or support frame assembly achieve substantially complete attenuation of pulsatile blood flow in the lumen of the tubular body of the graft 10, 210. In other embodiments, the assembled components of the support frame 16 or support frame assembly achieve up to 90% attenuation, or up to 75% attenuation, or up to 50% attenuation, or any percentage attenuation in between these ranges, with respect to pulsatile blood flow in the lumen of the tubular body 12, 212 of the graft 10, 210. In other embodiments, the assembled components of the support frame 16 or support frame assembly achieve sufficient or partial attenuation of pulsatile blood flow in the lumen 14, 214 of the tubular body 12, 212 of the graft 10, 210 so as to avoid or substantially delay intimal hyperplasia triggered by pulsatile blood flow. In accordance with this disclosure, any of the above-described support frame 16 configurations providing a pressure shock absorbing effect may be referred to as a “pressure shock absorber” or as a “pressure shock absorbing assembly.”

In terms of general effect, embodiments of vascular grafts provided with pulsation dampening characteristics, and corresponding methods of pulsation dampening in vascular grafts, utilize a change in cross-sectional configuration of the tubular body of the vascular graft to absorb kinetic energy from pulsatile blood flow in order to dampen the pressure pulses in order to lessen the effects of the pulsatile blood flow on the connection site between the vein and the AV graft, thereby avoiding, lessening and/or delaying the formation of intimal hyperplasia and/or other tissue reactions in the vein that may otherwise shorten the service life of an AV graft. In this way, the service life of an AV graft may be extended.

In some embodiments of this disclosure, pulsation dampening is achieved by reconfiguring a non-round cross-sectional configuration in a low pressure state of a tubular body possessing a wall of uniform thickness so that the cross-sectional configuration becomes round or at least more round in a high pressure state. In some embodiments of this disclosure, pulsation dampening is achieved by reconfiguring a non-round cross-sectional configuration in a low pressure state of a tubular body possessing a wall of non-uniform thickness so that the cross-sectional configuration becomes round or at least more round in a high pressure state. In some embodiments of this disclosure, pulsation damping is achieved by reconfiguring a round cross-sectional configuration in a low pressure state of a tubular body possessing a wall of non-uniform thickness so that the cross-sectional configuration becomes non-round in a high pressure state.

Although various non-limiting examples of the invention have been described in detail for the purpose of illustration based on what is currently considered to be the most practical and preferred aspects, it is to be understood that such detail is solely for that purpose and that the invention is not limited to the disclosed examples, but, on the contrary, is intended to cover modifications and equivalent arrangements that are within the spirit and scope of the appended claims. For example, it is to be understood that the present disclosure contemplates that, to the extent possible, one or more features of any example can be combined with one or more features of any other aspect or example.

The invention claimed is:

1. A vascular graft comprising:
an implantable tubular body defining a lumen; and
at least one support frame engaged to the tubular body for modifying a shape of a cross-section of at least a portion of the lumen,
wherein the tubular body and the at least one support frame are configured to receive pulsatile blood flow so that as pressure of blood flowing through the lumen increases, a cross-sectional area of the portion of the lumen increases while a perimeter of the portion of the lumen remains substantially unchanged in order to dampen pulsation of the blood flow.

2. The vascular graft of claim 1, wherein the tubular body and the at least one support frame are configured to transition between a first state, in which the portion of the lumen has a first cross-sectional area and a first perimeter and a second state, in which the portion of the lumen has a second cross-sectional area and a second perimeter, and wherein the second cross-sectional area is greater than the first cross-sectional area and the second perimeter is substantially equivalent to the first perimeter, and radial compliance of the tubular body permits transition between the first state and the second state.

3. The vascular graft of claim 2, wherein the tubular body and the at least one support frame are configured to be in the first state when pressure of fluid flowing through the lumen is diastolic pressure for a patient in which the vascular graft is implanted, and the tubular body and the at least one support frame are configured to transition to the second state when pressure of the fluid flowing through the lumen increases to systolic pressure for the patient in which the vascular graft is implanted.

4. The vascular graft of claim 2, wherein the tubular body and at least one support frame are configured to be in the first state when the pressure is less than a threshold pressure, and in the second state when the pressure is greater than or equal to the threshold pressure.

5. The vascular graft of claim 2, wherein in the first state, the shape of the cross-section of the lumen is elongated having a major dimension that is substantially greater than a minor dimension of the shape, and wherein in the second state, the shape of the cross-section of the lumen is substantially round with the minor dimension approaching or being substantially equivalent in length to the major dimension.

6. The vascular graft of claim 2, wherein in the first state, the shape of the cross-section of the lumen is an oval, and in the second state, the shape of the cross-section of the lumen is substantially circular.

7. The vascular graft of claim 2, wherein the second cross-sectional area is at least about 10% greater than the first cross-sectional area.

8. The vascular graft of claim 7, wherein the second perimeter is no more than about 5%, preferably about 2% to about 3%, greater than the first perimeter.

9. The vascular graft of claim 2, wherein the second cross-sectional area is from about 10% greater to about 15% greater than the first cross-sectional area.

10. The vascular graft of claim 1, wherein the tubular body and the at least one support frame are configured to dampen pulsation of the blood flow through the lumen of the tubular body compared to when no support frame is present.

11. The vascular graft of claim 10, wherein a pressure change for the pulsatile blood flow through the vascular graft is reduced by 5% to 20%, or by 5% to 50%, or by 20% to 50%, or by 20% to 90%, or by 50% to 90% compared to when no support frame is present.

12. The vascular graft of claim 1, wherein an outer surface of the tubular body comprises a low-friction material.

13. The vascular graft of claim 1, wherein the tubular body comprises at least one of ePTFE or silicone.

14. The vascular graft of claim 1, wherein the graft is an arterio-venous shunt graft.

15. The vascular graft of claim 1, wherein the at least one support frame comprises at least one of a band, a spring, a clip, or a helical coil configured to compress one or more portions of the tubular body when pressure of blood in the lumen is low, and wherein as the pressure of blood in the lumen increases these one or more portions of the tubular body expand radially outwardly against compression exerted by the at least one support frame.

16. The vascular graft of claim 1, wherein the at least one support frame comprises at least one of a band, a spring, a clip, or a helical coil configured to radially expand one or more portions of the tubular body when pressure of blood in the lumen is low causing the shape of the cross-section of the lumen to be oblong, and wherein as the pressure of blood in the lumen increases other portions of the tubular body expand radially outwardly causing the shape of the cross-section of the lumen to become more round.

17. The vascular graft of claim 1, wherein the at least one support frame comprises axially extending members forming an interconnected first helix and second helix.

18. The vascular graft of claim 1, wherein the at least one support frame comprises a heat-set metallic coil defining a lumen with an oval cross-section at body temperature, in an unbiased state, and configured to engage portions of an outer surface of the tubular body to compress the tubular body at body temperature.

19. The vascular graft of claim 18, wherein as pressure of blood flow through the lumen of the tubular body increases, the shape of the cross-section of the tubular body becomes more round, causing the cross-section of the lumen of the metallic coil to become more round.

20. The vascular graft of claim 1, wherein the at least one support frame comprises a heat-set metallic coil defining a lumen with a substantially circular cross-section at body temperature, in an unbiased state, and configured to engage portions of an outer surface of the tubular body to expand the tubular body at body temperature.

21. The vascular graft of claim 20, wherein as pressure of blood flow through the lumen of the tubular body increases, the shape of the cross-section of the tubular body becomes more round, causing a shape of the cross-section of the lumen of the metallic coil to become an almost round oval.

22. The vascular graft of claim 1, wherein the at least one support frame is fused to portions of an outer surface of the tubular body.

23. The vascular graft of claim 1, wherein the at least one support frame is fixedly connected to an outer surface of the tubular body by an adhesive.

24. The vascular graft of claim 1, wherein the at least one support frame connects to the tubular body along a major dimension of the tubular body, and wherein the outer surface of the tubular body is spaced apart from the at least one support frame at other portions of the outer surface.

25. The vascular graft of claim 1, wherein the at least one support frame comprises an elastomeric sleeve, and wherein the tubular body is inserted in and directly or indirectly connected to an inner surface of the elastomeric sleeve.

26. The vascular graft of claim 25, wherein the elastomeric sleeve defines a lumen, and wherein a shape of a cross-section of at least a portion of the lumen of the elastomeric sleeve is an oval at low pressure and becomes increasingly round as pressure increases in the lumen of the tubular body, which causes the cross-section of the lumen of the elastomeric sleeve to become round.

27. The vascular graft of claim 25, wherein the elastomeric sleeve is connected to the tubular body by at least one of adhesive bonding, solvent bonding, hot melt bonding, or sintering.

28. The vascular graft of claim 1, wherein the tubular body comprises an extruded tube and the at least one support frame comprises one or more polymeric layers fused to an outer surface of the tubular body.

29. The vascular graft of claim 1, wherein the tubular body comprises at least one raised ridge extending axially along at least a portion of the tubular body defining a channel of the lumen that spirals about a central longitudinal axis of the tubular body.

30. The vascular graft of claim 29, wherein the tubular body comprises multiple raised ridges extending axially along the portion of the tubular body defining multiple channels of the lumen.

31. The vascular graft of claim 4, wherein the threshold pressure is about 90 mmHg.

32. The vascular graft of claim 1, wherein the tubular body comprises an annular sidewall extending between a first end and a second end of the tubular body, and wherein, for at least a portion of the tubular body, a thickness of the sidewall varies about a periphery of the tubular body.

33. The vascular graft of claim 32, wherein thinner portions of the sidewall of the tubular body provide radial compliance for the tubular body.

34. The vascular graft of claim 1, wherein a sidewall of the tubular body has a non-concentric wall thickness where a central axis of the lumen is offset from a central axis of the tubular body.

35. A method of forming a vascular graft comprising the steps of:
attaching at least one support frame to a tubular body defining a lumen that modifies a shape of a cross-section of at least a portion of the lumen of the tubular body, wherein the tubular body and the at least one support frame are configured so that when a pressure of blood flowing through the lumen increases a cross-sectional area of the portion of the lumen increases while a perimeter of the portion of the lumen remains substantially unchanged; and
bending the tubular body or both the tubular body and the at least one support frame to form a vascular graft having substantially parallel substantially linear segments and a u-bend segment connecting the substantially parallel substantially linear segments.

36. The method of claim 35, wherein when the at least one support frame is attached to the tubular body, the tubular body and at least one support frame are configured to conform to a first state when pressure of blood flowing through the lumen is diastolic pressure for a patient in which the vascular graft is implanted, and the tubular body and at least one support frame are configured to transition to a second state when pressure of the blood flowing through the lumen increases to systolic pressure for the patient in which the vascular graft is implanted.

37. The method of claim 36, wherein the tubular body and the at least one support frame are configured to be in the first state when the pressure is less than a threshold pressure and in the second state when the pressure is greater than or equal to the threshold pressure.

38. The method of claim 37, wherein the threshold pressure is about 90 mmHg.

39. The method of claim 36, wherein a cross-sectional area of the lumen in the second state is about 10% greater to about 15% greater than the cross-sectional area of the lumen in the first state.

40. The method of claim 35, wherein the at least one support frame comprises at least one of a band, a spring, a clip, or a helical coil that, when attached to the tubular body, compresses one or more portions of the tubular body when pressure of blood in the lumen is low, and wherein as the pressure of blood in the lumen increases portions of the tubular body expand radially outwardly against a compression force exerted by the at least one support frame.

41. The method of claim 35, wherein the at least one support frame comprises at least one of a band, a spring, a clip, or a helical coil that, when attached to the tubular body, causes one or more portions of the tubular body to radially expand when pressure of blood in the lumen is low causing the shape of the cross-section of the lumen to be oblong, and wherein as the pressure of blood in the lumen increases, other portions of the tubular body expand radially outwardly causing the shape of the cross-section of the lumen to become more round.

42. The method of claim 35, wherein attaching the at least one support frame to the tubular body comprises fusing the at least one support frame to one or more portions of an outer surface of the tubular body.

43. The method of claim 35, wherein attaching the at least one support frame to the tubular body comprises fixedly attaching the at least one support frame to one or more portions of an outer surface of the tubular body by an adhesive.

44. The method of claim 35, wherein attaching the at least one support frame to the tubular body comprises attaching the at least one support frame to the tubular body at points along a major diameter of the tubular body, so that other portions of the at least one support frame are spaced apart from the outer surface of the tubular body.

45. The method of claim 35, further comprising attaching a first end of the vascular graft to a vein and a second end of the vascular graft to an artery to permit blood flow from artery to vein through the lumen of the tubular body, or attaching the first end of the vascular graft to an artery and the second end of the vascular graft to another artery to permit blood flow from artery to artery through the lumen of the tubular body.

46. The method of claim 45, wherein the vascular graft connected between the vein and the artery is configured for dialysis treatment.

47. A vascular graft comprising:
an implantable tubular body comprising an open first end, an open second end, and a sidewall extending between the first end and the second end, the sidewall defining a lumen,
wherein the sidewall of the tubular body comprises at least one raised ridge protruding radially outwardly relative to other portions of the sidewall of the tubular body and extending axially from the first end to the second end of the tubular body so as to define a channel of the lumen, and
wherein a flow path defined by the channel spirals about a central longitudinal axis of the tubular body.

48. The vascular graft of claim 47, further comprising at least one support frame connected to at least a portion of the sidewall of the tubular body that compresses at least a portion of the sidewall to form the at least one raised ridge.

49. The vascular graft of claim 47, wherein the tubular body comprises multiple raised ridges extending axially along the tubular body so as to define multiple channels of the lumen.

50. The vascular graft of claim 49, wherein the multiple ridges and channels are positioned to impart a spiral flow for blood flowing through the tubular body.

51. The vascular graft of claim 49, wherein flow paths defined by each of the multiple channels complete at least one full rotation about the perimeter of the tubular body between the first end and the second end of the tubular body.

52. The vascular graft of claim 47, wherein in a first state, a shape of the cross-section of the lumen is a star having points and recessed portions, and as pressure of blood flowing through the lumen increases, the vascular graft transitions to a second state in which the recessed portions of the star move radially outwardly away from the central longitudinal axis of the tubular body, thereby causing the shape of the cross-section of the lumen to become more round.

53. The vascular graft of claim 52, wherein portions of the tubular body forming recessed portions of the star-shaped lumen are more flexible than portions of the tubular body forming points of the star-shaped lumen.

54. The vascular graft of claim 52, wherein portions of the tubular body forming recessed portions of the star-shaped lumen are thinner than portions of the tubular body forming points of the star-shaped lumen.

55. The vascular graft of claim 52, wherein the tubular body is in the first state when pressure of blood flowing through the lumen is less than a threshold blood pressure and in the second state when the pressure of blood is greater than the threshold blood pressure.

56. The vascular graft of claim 55, wherein the threshold blood pressure is about 90 mmHg.

57. The vascular graft of claim 52, wherein a cross-sectional area of the lumen when the graft is in the second state is from about 10% greater to about 15% greater than the cross-sectional area of the lumen when the graft is in the first state.

58. The vascular graft of claim 47, wherein, for at least a portion of the tubular body, a thickness of the sidewall varies about a periphery of the tubular body.

59. The vascular graft of claim 58, wherein thinner portions of the sidewall of the tubular body provide radial compliance for the tubular body.

60. The vascular graft of claim 47, wherein the sidewall of the tubular body has a non-concentric wall thickness where a central axis of the lumen is not co-extensive with a central axis of the tubular body.

61. A vascular graft comprising:
an implantable tubular body comprising an open first end, an open second end, and a sidewall extending between the first end and the second end, the sidewall defining a lumen,
wherein the tubular body is configured such that as pressure of blood flowing through the lumen increases a cross-sectional area of a portion of the lumen increases while a perimeter of the portion of the lumen remains substantially unchanged.

62. The vascular graft of claim 61, wherein the tubular body is configured to transition between a first state in which the portion of the lumen has a first cross-sectional area and a first perimeter, and a second state in which the portion of the lumen has a second cross-sectional area and a second perimeter, and wherein the second cross-sectional area is greater than the first cross-sectional area and the second perimeter is substantially equivalent to the first perimeter, and radial compliance of the tubular body permits transition between the first state and the second state.

63. The vascular graft of claim 62, wherein the tubular body is configured to be in the first state when pressure of blood flowing through the lumen is diastolic pressure for a patient in which the vascular graft is implanted, and the tubular body is configured to transition to the second state when pressure of the blood flowing through the lumen increases to systolic pressure for the patient in which the vascular graft is implanted.

64. The vascular graft of claim 62, wherein the tubular body is in the first state when the pressure of the blood is less than a threshold pressure and in the second state when the pressure of the blood is greater than or equal to the threshold pressure.

65. The vascular graft of claim 64, wherein the threshold blood pressure is about 90 mmHg.

66. The vascular graft of claim 62, wherein in the first state, the shape of the cross-section of the lumen is selected from the group consisting of a triangle, star, diamond, rectangle, square, polygon, trapezoid, crescent, oval, or elongated pill shape.

67. The vascular graft of claim 62, wherein in the first state, the shape of the cross-section of the lumen is an irregular shape.

68. The vascular graft of claim 61, wherein the tubular body comprises at least one raised ridge extending axially along at least a portion of the tubular body so as to define a channel of the lumen that spirals about a central longitudinal axis of the tubular body.

69. The vascular graft of claim 68, wherein the tubular body comprises multiple raised ridges extending axially along the tubular body so as to define multiple channels of the lumen.

70. The vascular graft of claim 61, wherein, for at least a portion of the tubular body, a thickness of the sidewall varies about a periphery of the tubular body.

71. The vascular graft of claim 70, wherein thinner portions of the sidewall of the tubular body provide radial compliance for the tubular body.

72. The vascular graft of claim 61, wherein the sidewall of the tubular body has a non-concentric wall thickness where a central axis of the lumen is not co-extensive with a central axis of the tubular body.

73. A vascular graft comprising:
an implantable tubular body having a wall defining a lumen, wherein the wall has a variable non-concentric wall thickness and includes at least one thin portion and at least one thick portion, wherein the tubular body is configured to receive pulsatile blood flow so that as pressure of blood flowing through the lumen increases, a cross-sectional area of the lumen increases while a perimeter of the lumen remains substantially unchanged in order to dampen pulsation of the blood flow.

74. The vascular graft of claim 73, wherein the at least one thin portion possesses about 10-90% of the thickness of the at least one thick portion of the wall, or the thin portion possesses about 20-80% of the thickness of the at least one thick portion of the wall, or the at least one thin portion possesses about 10-50% of the thickness of the at least one thick portion of the wall.

75. The vascular graft of claim 73, wherein when in a relaxed low pressure state a cross-sectional configuration of the tubular body has a crescent moon shape and when in a flexed high pressure state the cross-sectional configuration of the tubular body has a round or almost round shape.

76. A vascular graft comprising:
an implantable tubular body having a wall defining a lumen, wherein the wall has a variable wall thickness and includes at least one thin portion and at least one thick portion,
wherein the tubular body is configured to receive pulsatile blood flow so that as pressure of blood flowing through the lumen increases, the at least one thin portion of the wall moves radially outward in order to dampen pulsation of the pulsatile blood flow.

77. The vascular graft of claim 76, wherein the implantable tubular body is configured to adopt an out-of-round cross-sectional shape as the at least one thin portion of the wall moves radially outward, thereby dampening the pulsation of the blood flow.

78. The vascular graft of claim 76, wherein the implantable tubular body comprises a first thin portion on a first side of the wall and a second thin portion on an opposing side of the wall.

79. The vascular graft of claim 76, wherein the implantable tubular body is configured to transition from a relaxed low pressure state to a high pressure state, and wherein a cross-sectional shape of a lumen defined by the tubular body becomes less round as the tubular body transitions from the relaxed low pressure state to the high pressure state.

80. The vascular graft of claim 79, wherein the cross-sectional shape of the lumen in the relaxed low pressure state is substantially circular and the cross-sectional shape in the high pressure state is substantially an oval shape.

81. The vascular graft of claim 76, wherein the at least one thin portion possesses about 10-90% of the thickness of the at least one thick portion of the wall, or the thin portion possesses about 20-80% of the thickness of the at least one thick portion of the wall, or the at least one thin portion possesses about 10-50% of the thickness of the at least one thick portion of the wall.
